(19) United States
(12) Patent Application Publication
(10) Pub. No.: US 2024/0415885 A1
(43) Pub. Date: Dec. 19, 2024

(54) Method for the generation of genetically modified NK cells

(71) Applicant: MILTENYI BIOTEC B.V. & CO. KG, Bergisch Gladbach (DE)

(72) Inventors: Congcong Zhang, Bergisch Gladbach (DE); Melissa Quadflieg, Bergisch Gladbach (DE); Nina Moker, Bergisch Gladbach (DE)

(73) Assignee: MILTENYI BIOTEC B.V. & CO. KG, Bergisch Gladbach (DE)

(21) Appl. No.: 18/701,572

(22) PCT Filed: Oct. 13, 2022
(86) PCT No.: PCT/EP2022/078470

§ 371 Date: Apr. 15, 2024

(30) Foreign Application Priority Data
Oct. 15, 2021 (EP) 21202832.8

(51) Int. Cl. A61K 35/17; A61K 39/00; C07K 14/735; C07K 16/28; C12N 5/0783; C12N 9/22; C12N 15/11; C12N 15/86; C12N 15/87

(52) U.S. Cl. CPC A61K 35/17; A61K 39/4613; A61K 39/4631; A61K 39/4632; A61K 39/464411; C07K 14/70535; C07K 16/283; C12N 5/0646; C12N 9/22; C12N 15/111; C12N 15/86; C12N 15/87; C12N 2310/20

(57) ABSTRACT

The present invention provides an in-vitro method for the generation of a population of genetically modified natural killer (NK) cells comprising the steps in the following order: a) obtaining a sample comprising NK cells and other cells, b) enrichment of NK cells from said sample, c) introducing a genetic modifier 1 into said NK cells by electroporation, d) introducing a genetic modifier 2 into said NK cells by transduction, e) expanding said genetically modified NK cells, thereby generating a population of genetically modified NK cells.

### PRIOR APPLICATION

This application is the § 371 U.S. national stage of international application PCT/EP2022/078470, filed Oct. 13, 2022, published as WO 2023/062113 on Apr. 20, 2023; which claims the priority benefit of European application 21202832.8, filed Oct. 15, 2021. The PCT application is hereby incorporated herein by reference in its entirety for all purposes.

### FIELD OF THE INVENTION

The present invention generally relates to the field the generation of genetically modified NK cells, in particular to the generation of genetically modified NK cells by a method using first a step of electroporation followed by a step of transduction to introduce genetic modifiers into said NK cells.

### BACKGROUND OF THE INVENTION

Natural killer (NK) cells are cytotoxic innate lymphocytes and play a vital role in immune surveillance against viral infection and malignancies (Björkström. N. K. et al (2021). Nat. Rev. Immunol. 11, 1-12; Huntington, N. D. et al (2020). Nat. Rev. Cancer. 20, 437-454). The activation of NK cells depends on their germ-line encoded activating and inhibitory receptors, therefore they can rapidly recognize and eliminate transformed cells without prior sensitization. Clinical evidence has shown that donor-derived NK cells have low risk in inducing graft-versus-host-disease (GvHD), thus making them ideal for using as “off-the-shelf” cell therapeutics in allogeneic settings. Major advances have been made in harnessing NK cells in cancer immunotherapy in recent years. To enhance their effector functions, NK cells can be genetically modified by viral transduction to express transgenes such as chimeric antigen receptors (CARs) or cytokines. More recently, nonviral genome editing technologies, such as transposons, endonucleases and base editors, have been applied to precisely alter DNAs in NK cells.

Although NK cells are notoriously difficult to common gene delivery methods due to their cell intransigence to genetic modification, significant progress has recently been made to overcome the delivery challenges posed to NK cells. NK cell transduction has been achieved by using retroviral vectors with feeder cell activation (Streltsova, M. A. et al (2017). J. Immunol. Methods 450, 90-94; Liu, E. et al, (2018). Leukemia 32, 520-531) or Baboon envelope (BaEV) pseudotyped lentiviral vectors (LVs) without using feeder cells (Girard-Gagnepain, A. et al (2014). Blood 124, 1221-1231; Bari et al. (2019). Front. Immunol. 10, 2001). Meanwhile, efforts have also been made to optimize NK cell electroporation (Rautela et al (2018). Preprint; Ingegnere et al (2019). Front. Immunol. 10, 957). Since both methods hold advantages over each other in particular applications, it thereby offers great potentials to combine viral transduction and electroporation to generate gene-modified NK cells with superior functionality for immunotherapy. However, little success has been reported in this direction so far for NK cells. It might be partly because, both viral transduction and electroporation are highly stressful and stress accumulated from both processes may markedly increase the risk to cause irreversible damage of NK cells.

Basar et al. (Blood Adv. 4, 5868-5876.) proposed a workflow to generate CRISPR modified CAR NK cells for cancer therapy. In the proposed workflow, NK cells will be transduced with a viral vector to express CARs followed by CRISPR genome editing by electroporation (but no data were shown).

Daher et al. (Blood 137, 624-636.) described a protocol to generate gene-editing CAR NK cells by transducing cord-blood derived NK cells with a CD19 CAR with retroviral vectors on day 4 after isolation, followed by electroporation with CISH CRISPR RNPs on day 7. The gene-modified NK cells were weekly stimulated with K562 feeder cells expressing membrane-bound IL-21, 4-1BB ligand and CD48. The final cell products were harvested on day 21 of culture. However, the gene modification can only be finished 7 days after NK cell isolation and gene-modified NK cell proliferation had to be optimized by using feeder cells, which are based on an immortal cell line established from a patient with chronic myelogenous leukemia.

There is a need in the art for an improved or alternative method for the generation of genetically modified NK cells that allow e.g. the realization of full therapeutic potentials of NK cells in clinical applications.

### BRIEF DESCRIPTION OF THE INVENTION

The inventors surprisingly found that the herein disclosed method for the generation of a population of genetically engineered NK cells posed no severe stress to the NK cells although these NK cells underwent both processes electroporation and transduction. The herein disclosed method results in expansion rates that are comparable to expansion rates of the NK cells that received electroporation only. It is essential for the process as disclosed herein that the step of electroporation of NK cells is performed before the step of transduction is performed. In addition, surprisingly it was found that the process as disclosed herein works best in the time frames for electroporation and transduction of the NK cells as disclosed herein.

Furthermore, the method as disclose leads to high transduction and electroporation efficiency of NK cells. Meanwhile, NK cells treated with said method were fully functional and exhibited high anti-tumor activity and in vitro proliferation rate.

Surprisingly, NK cells treated with the herein disclosed method possess higher transgene expression, compared with NK cells engineered with the method by transduction prior to electroporation or NK cells transduced only.

Even more unexpectedly, NK cells treated present method express higher level of CD16, compared with NK cells engineered with the TD-Elpo method or NK cells transduced only (herein it is referred to “Elpo-TD method”, if the electroporation step is before the transduction step within the method, and to “TD-Elpo method”, if the transduction step is before the electroporation step within the method).

Unexpectedly, NK cells treated with the present method displayed higher expansion potential in vitro, compared with NK cells engineered with the method by transduction prior to electroporation of NK cells.

The present invention also provides a population of genetically engineered NK cells obtained by the method as disclosed herein. Said population has a higher expression of the transgene such as a CAR or TCR on the cell surface of the NK cells and/or a higher expression of CD16 as compared to populations known in the art.

CD16, also known as FcγRIIIa, is an activating receptor that binds to Fc portion of antibodies to induce antibody-dependent cell-mediated cytotoxicity (ADCC) of NK ceils against tumor cells or viral infected cells. Tumor targeting therapeutic antibodies, such as Rituximab (specific for CD20), Trastuzumab (specific for HER2/ErbB2) or Daratumumab (specific for CD38), have been broadly applied in the clinic with great success. NK cells are critical to clinical responses of therapeutic antibodies by recognizing and eliminating antibody-coated malignant cells through CD16 ligation. It has been demonstrated that increased CD16 expression positively correlated with the degree of ADCC of NK cells and thereby enhances NK cell clinical efficacy (Koehn et al., 2012, Front. Pharmacol. 3, 91; Peruzzi et al., 2013 J. Immunol. 191, 1883-1894).

### BRIEF DESCRIPTION OF THE DRAWINGS

FIG. 1. Schematic representations of the in vitro Electroporation-Transduction (Elpo-TD) and Transduction-Electroporation (TD-Elpo) methods for the generation of a population of genetically modified natural killer (NK) cells.

FIG. 2A. Flow cytometric analysis of CD33 expression at day 13 of in vitro expansion on the surface of CD33-KO CAR-NK cells generated by the Elpo-TD method or TD-Elpo method. Unmodified wild-type NK (WT-NK) cells, NK cells only receiving electroporation of CRISPR/Cas RNPs targeting cd33 at the corresponding time points (CD33-KO NK) served as controls.

FIG. 2B. Comparison of CD33 gene knockout (KO) efficiencies among the gene-modified NK cells shown in FIG. 2A.

FIG. 2C. Flow cytometric analysis of CD33-specific chimeric antigen receptor (CD33 CAR) expression at day 13 of in vitro expansion on the surface of CD33-KO CAR-NK cells generated by the Elpo-TD method or TD-Elpo method shown in FIG. 2A. Unmodified wild-type NK (WT-NK) cells and NK cells only receiving transduction with CD33 CARs (CAR-NK) served as controls.

FIG. 2D. CD33 CAR expression on the surface of the gene-modified NK cells shown in FIG. 2C.

FIG. 2E. Flow cytometric analysis of CD16 expression at day 13 of in vitro expansion on the surface of CD33-KO CAR-NK cells generated by the Elpo-TD method or TD-Elpo method shown in FIG. 2C. Unmodified wild-type NK (WT-NK) cells and NK cells only receiving transduction with CD33 CARs (CAR-NK) served as controls.

FIG. 2F. Expression of CD16 on the surface of the gene-modified NK cells shown in FIG. 2E.

FIG. 3A. Expression of CD33 on the surface of established RS4;11 human B cell precursor acute lymphoblastic leukemia (B-ALL) cells ectopically expressing human CD33 (CD33-positive RS4;11). RS4;11 cells without ectopic human CD33 expression (CD33-negative RS4;11) served as controls.

FIG. 3B. CAR-mediated cytotoxicity of CD33-KO CAR-NK cells specific for CD33 generated by the Elpo-TD or TD-Elpo method against CD33-positive RS4;11 human B cell precursor acute lymphoblastic leukemia (ALL) cells in a 4-hour co-culture cytotoxicity assay at the indicated effector to target cell (E:T) ratios. E:T ratios were calculated based on CAR-NK cells. Total NK cell numbers were theoretically calculated according to the CAR transduction efficiencies.

FIG. 4A. In vitro proliferation of CD33-KO CAR-NK cells specific for CD33 generated by the Elpo-TD or TD-Elpo method over a period of 15 days after NK cell enrichment. NK cells only received electroporation of CRISPR/Cas RNPs targeting cd33 gene at the corresponding time points (CD33-KO NK) served as controls.

FIG. 4B. The cell counts of the CAR-modified NK cells generated by the Elpo-TD or TD-Elpo method shown in FIG. 4A were compared for the endpoint of the time series (day 15).

FIG. 5A. CD33 CAR and CD16 surface expression on CAR-NK cells generated by the Elpo-TD method or TD-Elpo method with mock electroporation and CD33 CAR transduction were demonstrated by flow cytometric analysis at day 13 of in vitro expansion.

FIG. 5B. In vitro proliferation of CD33 CAR-NK cells generated by the Elpo-TD or TD-Elpo method with mock electroporation and CD33 CAR transduction shown in FIG. 5A over a period of 27 days after NK cell enrichment.

FIG. 5C. CAR-mediated cytotoxicity of CD33 CAR-NK cells generated by the Elpo-TD method against CD33-positive and CD33-negative RS4;11 human B cell precursor acute lymphoblastic leukemia (ALL) cells in a 4-hour co-culture cytotoxicity assay at the indicated effector to target cell (E:T) ratios. E:T ratios were calculated based on CAR-NK cells.

FIG. 6A. NKG2A and CD33 CAR surface expression on NKG2A-KO CAR-NK cells generated by the Elpo-TD method were demonstrated by flow cytometric analysis at day 13 of in vitro expansion. Unmodified wild-type NK (WT-NK) cells and mock-electroporated, CD33 CAR-transduced NK cells (Mock-KO CAR-NK) served as controls.

FIG. 6B. Comparison of NKG2A gene knockout (KO) efficiencies between Mock-KO CAR-NK cells and NKG2A-KO CAR-NK cells shown in FIG. 6A.

FIG. 6C. Cytotoxicity of Mock-KO CAR-NK cells and NKG2A-KO CAR-NK cells shown in FIG. 6A against CD33-positive and CD33-negative RS4;11 human B cell precursor acute lymphoblastic leukemia (ALL) cells in a 4-hour co-culture cytotoxicity assay at the indicated effector to target cell (E:T) ratios. E:T ratios were calculated based on CAR-NK cells.

FIG. 7. Growth of NK cells in culture after isolation. Viable NK cells were counted at the indicated time points. Data shown are from two separate experiments with NK cells from two different donors.

FIG. 8. Determination of activation status of freshly isolated or overnight cultured NK cells. NK cells were isolated with either positive or negative selection method. The isolated NK cells were either used immediately or cultured overnight at 37° C. in an incubator. The expression of the early activation markers of CD25 and CD69 on the NK cells was determined by flow cytometric analysis.

FIG. 9A. NKG2A surface expression on the gene-modified NK cells generated by the Elpo-TD method by performing electroporation with either freshly isolated NK cells (0 hour (0 h) after preparation of the sample and direct subsequent NK cell enrichment) or overnight-cultured NK cells (18 hours (18 h) after preparation of the sample and direct subsequent NK cell enrichment). NK cells were electroporated with NKG2A-specific CRISPR RNPs (NKG2A-KO) or without any RNPs (Mock-KO), followed by viral transduction with a BDCA2-specific CAR construct (BDCA2 CAR). The NKG2A expression was demonstrated by flow cytometric analysis at day 13 of in vitro expansion. Unmodified wild-type NK (WT-NK) cells and overnight-cultured NK cells only electroporated with NKG2A-specific CRISPR RNPs 18 hours after preparation of the sample and direct subsequent NK cell enrichment (NKG2A-KO (18 h) NK) served as controls.

FIG. 9B. CAR surface expression on the gene-modified NK cells shown in FIG. 9A.

FIG. 9C. Flow cytometry dot plots of NKG2A and CAR surface expression on the gene-modified NK cells shown in FIGS. 9A and 9B.

FIG. 9D. Cytotoxicity of the gene-modified NK cells shown in FIG. 9A against BDCA2-positive RS4;11 human B cell precursor acute lymphoblastic leukemia (ALL) cells in a 16-hour co-culture cytotoxicity assay at the indicated effector to target cell (E:T) ratios. E:T ratios were calculated based on CAR-NK cells.

FIG. 10A. NKG2A surface expression on NKG2A-KO CAR-NK cells generated by the Elpo-TD method by performing electroporation with NKG2A-specific CRISPR RNPs at different time points (from 0 hour (0 h) to 144 hours (144 h) after preparation of the sample and direct subsequent NK cell enrichment), followed by viral transduction with a CD33-specific CAR construct. Unmodified wild-type NK (WT-NK) cells and NK cells only transduced with the CD33-specific CAR construct 48 hours after preparation of the sample and direct subsequent NK cell enrichment (CAR-NK) served as controls. Data shown are from two separate experiments with NK cells from two different donors. Mean values±SD are shown; n=2.

FIG. 10B. CAR surface expression on NKG2A-KO CAR-NK cells shown in FIG. 10A. Mean values±SD are shown; n=2.

FIG. 10C. Flow cytometry dot plots of NKG2A and CAR surface expression from one representative experiment shown in FIGS. 10A and 10B.

FIG. 10D. Cytotoxicity of the gene-modified NK cells shown in FIG. 10A against CD33-positive OCI-AML2 acute myeloid leukemia (AML) cells in a 18-hour co-culture cytotoxicity assay at a target cell (E:T) ratio of 1:1. E:T ratio was calculated based on CAR-NK cells. Mean values±SD are shown; n=2.

FIG. 11A. NKG2A surface expression on NKG2A-KO CAR-NK cells generated by the Elpo-TD method with different time intervals between electroporation and transduction. The isolated NK cells were electroporated 18 hours (18 h) after preparation of the sample and direct subsequent NK cell enrichment with NKG2A-specific CRISPR RNPs, followed by viral transduction (TD) with a CD33-specific CAR construct 24 hours (24 h), 48 hours (48 h), 66 hours (66 h) or 138 hours (138 h) after preparation of the sample and direct subsequent NK cell enrichment. Unmodified wild-type NK (WT-NK) cells and NK cells only electroporated 18 hours (18 h) after preparation of the sample and direct subsequent NK cell enrichment with NKG2A-specific CRISPR RNPs (NKG2A-KO) or without any RNPs (Mock-KO) served as controls. Data shown are from two separate experiments with NK cells from two different donors. Mean values±SD are shown; n=2.

FIG. 11B. CAR surface expression on NKG2A-KO CAR-NK cells shown in FIG. 11A.

FIG. 11C. Flow cytometry dot plots of NKG2A and CAR surface expression from one representative experiment shown in FIGS. 11A and 11B.

### DETAILED DESCRIPTION OF THE INVENTION

In a first aspect the present invention provides an in-vitro method for the generation of a population of genetically modified natural killer (NK) cells comprising the steps in the following order
a) obtaining (providing) a sample comprising NK cells and other cells
b) enrichment of NK cells from said sample
c) introducing a genetic modifier 1 into said NK cells by electroporation
d) introducing a genetic modifier 2 into said NK cells by transduction
e) expanding said genetically modified NK cells,
thereby generating a population of genetically modified NK cells.

Said sample may be whole blood of a human, a leukapheresis of a subject, buffy coat, PBMC, outgrown or isolated NK cells or cells from an in-vitro cell culture.

Said method, wherein said method comprises before said enrichment of NK cells a step of preparation of the sample comprising NK cells and other cells by centrifugation.

Said preparation of said sample may result in volume reduction, rebuffering, removal of serum, erythrocyte reduction, platelet removal, and/or washing.

Said method, wherein said method comprises after said enrichment of NK cells but before introducing said genetic modifier 1 into said NK cells by electroporation the step of activation of NK cells, e.g. by a modulatory agent. Said modulatory agent (or NK cell activation agent) may be a molecule such as a particle, bead or nanomatrix that has coupled thereto one or more stimulatory agents that provide activation signals to NK cells such as stimulating antibodies against CD2 and CD335. An example for such an NK cell activation reagent is Miltenyi Biotec's NK Cell Activation/Expansion Kit (Miltenyi Biotec GmbH, Bergisch Gladbach, Germany, Order no. 130-094-483) or the nanomatrix described in EP2824112B1.

Alternatively or additionally, said modulatory agents may be cytokines or other antibody such as IL-2, IL-15, IL-18, IL-1 family, IL-12, CCL5, IL-21, type I interferons such as IFNα, and/or IFNγ.

Said method, wherein said enriched NK cells are not activated prior to introducing said genetic modifier 1 into said NK cells by electroporation.

Obtaining a sample comprising NK cells and other cells (or providing said sample) may be the provision of a sample provided/obtained from a subject such as a human. Said sample may be whole blood of a human, a leukapheresis of a subject, buffy coat, PBMC, outgrown or isolated NK cells or cells from an in-vitro cell culture.

Alternatively said method as disclosed herein may start with step “enrichment of NK cells from a sample comprising NK cells and other cells” followed by the other steps as disclosed herein.

In one embodiment of the present invention the method may be an in-vitro method for the generation of a population of genetically modified natural killer (NK) cells comprising the steps in the following order
a) enrichment of NK cells from a sample comprising NK cells and other cells
b) introducing a genetic modifier 1 into said NK cells by electroporation
c) introducing a genetic modifier 2 into said NK cells by transduction
d) expanding said genetically modified NK cells,
thereby generating a population of genetically modified NK cells.

Said method, wherein said NK cells are human NK cells.

Said enrichment of NK cells may be performed by enrichment of CD56+ cells followed by depletion of CD3+ cells from said sample. Alternatively said enrichment of NK cells may be the CD3 and CD19 depletion of cells from said sample. Alternatively, said enrichment may be the depletion of CD3+ cells followed by the enrichment of CD56+ cells from said sample.

The NK cells may be further enriched for CX3CR1 negative NK cells from a population or sample comprising CX3CR1 positive and CX3CR1 negative NK cells.

Said enrichment of CX3CR1 negative NK cells or CX3CR1 negative NK cell subsets/subpopulations may be performed by depletion of the CX3CR1 negative cells in a separation process from a sample or population comprising CX3CR1 positive and CX3CR1 negative NK cells, wherein the depleted fraction is the target fraction that comprises the CX3CR1 negative cells, e.g. by separation methods such as cell separation methods, e.g. MACS® (Miltenyi Biotec GmbH) or fluorescence activated cell sorting such as flow cytometry using anti-CX3CR1 antibodies or fragments thereof.

Said method, wherein said generated population of genetically modified NK cells expresses a higher level of CD16 as compared to a population of genetically engineered NK cells that is obtained by the method of first performing the transduction process of introducing the genetic modifier 2 into the NK cells followed by the electroporation process of introducing the modifier 1 into the NK cells.

Said higher level of expression of CD16 may be at least 10% higher, 15% higher, 20% higher or 25% higher.

Said method, wherein said electroporation is performed one day after enrichment of said NK cells, and wherein said transduction is performed one day after said electroporation.

The term “one day after said electroporation” may comprise 24 hours+/−12 hours after said electroporation, more preferentially, the term “one day after said electroporation” may comprise 24 hours+/−6 hours after said electroporation.

The term “one day after enrichment of said NK cells” may comprise 24 hours+/−12 hours after said enrichment, more preferentially, the term “one day after enrichment of said NK cells” may comprise 24 hours+/−6 hours after said enrichment.

Said method, wherein said electroporation is performed, between 0 hours (i.e. directly) and 144 hours, between 0 hours and 120 hours, between 0 hours and 96 hours, between 0 hours and 72 hours, between 0 hours and 48 hours, between 6 hours and 144 hours, between 6 hours and 120 hours, between 6 hours and 96 hours, between 6 hours and 72 hours, between 6 hours and 48 hours, between 12 hours and 36 hours, between 18 hours and 30 hours, or between 18 hours and 24 hours after preparation of said sample, and wherein said transduction is performed between 6 hour and 144 hours, between 6 hours and 120 hours, between 6 hours and 96 hours, between 6 hours and 72 hours, between 6 hours and 48 hours, between 12 hours and 36 hours, between 18 hours and 30 hours, or between 18 hours and 24 hours after said electroporation.

As shown in Example 6 there is no expansion of the enriched NK cells in the first 3 days of culturing the cells.

Said method, wherein the NK cells do not expand prior to introducing said genetic modifier 1 into said NK cells by electroporation.

Said method, wherein the NK cells do not expand prior to introducing said genetic modifier 1 into said NK cells by electroporation, and wherein said electroporation is performed between 0 hours and 72 hours.

Said method, wherein the NK cells are activated but do not expand prior to introducing said genetic modifier 1 into said NK cells by electroporation.

Said method, wherein the NK cells are not activated and do not expand prior to introducing said genetic modifier 1 into said NK cells by electroporation.

It was surprisingly found that the transduction is worse if the transduction is performed later than 120 hours after the electroporation of the sample.

Said method, wherein the time interval (window) between said electroporation and said transduction is 6 hours, 30 hours, 48 hours, or 120 hours, and wherein said electroporation is at 0 hours, 18 hours, 48 hours and 144 hours after said enrichment and/or said preparation, and wherein said transduction is not before 24 hours after said preparation and/or said enrichment.

It was surprisingly found that electroporation at 0-48 hours after preparation of the sample and/or enrichment of the NK cells (normally enrichment of the NK cells directly follows the preparation of the sample) showed higher gene knockout (KO) efficiency and CAR expression, compared with electroporation at 144 hours (see Example 9) and that transduction at 6-48 hours after electroporation was better than transduction at 120 hours after electroporation (Example 10).

Said method, wherein said electroporation is performed between 0 hours and 48 hours after said preparation of the sample and/or said enrichment of the NK cells, and wherein said transduction is performed between 6 hour and 48 hours after said electroporation.

Said method, wherein said electroporation of the NK cells is performed about 18 hours after said preparation of the sample and/or said enrichment of NK cells, and wherein said transduction is performed about 6 hours after said electroporation.

Said method, wherein said electroporation is performed about 18 hours after said preparation of the sample and/or said enrichment of the NK cells, and wherein said transduction is performed about 30 hours after said electroporation.

Said method, wherein said electroporation of the NK cells is performed about 18 hours after said preparation of the sample and/or said enrichment of NK cells, and wherein said transduction is performed about 48 hours after said electroporation.

Said method, wherein said electroporation is performed about 0 hours after said preparation of the sample and/or said enrichment of the NK cells, and wherein said transduction is performed about 48 hours after said electroporation.

Said method, wherein said genetic modifier 1 is DNA, RNA, protein, or a combination thereof able to selectively engineer at least one target gene by DNA cleavage, and/or wherein said genetic modifier 2 is a viral vector comprising at least one transgene.

Said genetic modifier 1 may be DNA, RNA, protein or a combination thereof able to selectively engineer at least one target gene by DNA cleavage resulting in inactivation (or knock out), correction or insertion (knock in) of said at least one target gene.

In one embodiment of the invention said genetic modifier 1 may be DNA, RNA, protein or a combination thereof able to selectively engineer at least one target gene by DNA cleavage resulting in inactivation of the at least one target gene by knock-out of said at least one target gene.

In one embodiment of the invention said genetic modifier 1 may be DNA, RNA, protein or a combination thereof able to selectively engineer at least one target gene by DNA cleavage resulting in insertion of the at least one target gene by knock-in of said at least one target gene.

In one embodiment of the invention said genetic modifier 1 may be DNA, RNA, protein or a combination thereof able to selectively engineer at least one target gene by DNA cleavage resulting in correction of the at least one target gene by substitution of at least a single nucleotide of said at least one target gene.

Said at least one target gene may be a coding nucleic acid sequence of a gene within the genome of a subject or a regulatable element of such a gene.

Said genetic modifier 1 may be an engineered nuclease selected from the group consisting of a meganuclease, a zinc-finger nuclease (ZFN), a transcription activator-like effector nuclease (TALE-Nuclease), a CRISPR/Cas nuclease, MAD nuclease, CRISPR/Cpf1, Cas12-type-derived nucleases and a megaTAL nuclease.

Said MAD nuclease may be e.g. MAD2, MAD7, or MAD70.

Said viral vector comprising at least one transgene may be a retroviral vector comprising a transgene. Said retroviral vector may be a lentiviral vector.

Said method, wherein the method may generate at day 15 of the process at least 2 times more genetically modified NK cells in said population as compared to the method of first performing the transduction process of introducing the genetic modifier 2 into the NK cells followed by the electroporation process of introducing the modifier 1 into the NK cells, wherein in both methods the starting amount of NK cells in said sample may be the same.

Day 2 of the process may be the day when the NK cells underwent the transduction.

Said method, wherein said at least one transgene is a chimeric antigen receptor (CAR) or a T-cell receptor (TCR).

Said method, wherein said generated population of genetically modified NK cells expresses a higher level of said at least one transgene compared to a population of genetically engineered NK cells that is obtained by the method of first performing the transduction process of introducing the genetic modifier 2 into the NK cells followed by the electroporation process of introducing the modifier 1 into the NK cells.

Said higher level of expression of said at least one transgene (on the surface of the genetically engineered NK cells) may be at least 10% higher, 15% higher or 20% higher.

Said at least one transgene may be a chimeric antigen receptor (CAR) or TCR specific for CD3, CD4, CD5, CD7, CD8, CD19, CD20, CD22, CD38, BCMA, CD33, CD123, BDCA2, CD56, receptor tyrosine kinase-like orphan receptor 1 (ROR1), ganglioside GD2, ganglioside GD3, c-met, CD30, folate binding protein, HER2/ErbB2, EGFR, EGFRvIII, VEGFR2, PSMA, CS1 (also known as SLAMF7), CLEC12A, FLT-3, CD79a, CD79b, CD179b, mesothelin, CD10, CD34, EpCAM, CD66, IL13RA2, CD171, B7H3 (CD276), KIT (CD117), Carcinoembryonic antigen (CEA), Interleukin 11 receptor alpha (IL-11Ra), claudin 6 (CLDN6), prostate stem cell antigen (PSCA), Lewis(Y) antigen; CD24; Platelet-derived growth factor receptor beta (PDGFR-beta); Stage-specific embryonic antigen-4 (SSEA-4); Folate receptor alpha, fibroblast activation protein alpha (FAP), CD97, mammary gland differentiation antigen (NY-BR-1), Wilms tumor protein (WT1), Cancer/testis antigen 1 (NY-ESO-1), tumor protein p53 (p53), p53 mutant, Rat sarcoma (Ras) mutant, human papilloma virus E6 (HPV E6), human papilloma virus E7 (HPV E7), Glypican-3 (GPC3), kappa chain, lambda chain, CD99 or CCR4.

Transgenes may also be cytokines such as IL-15, IL-15 receptors, IL-15/IL-15R fusion proteins or other genes encoding for stimulatory or regulatory proteins such as high-affinity CD16, NKG2D, NKp46, CD2, growth regulation factors or differentiation regulation factors.

Transgenes may be one or more CARs or TCRs combined with above-mentioned genes. Said method, wherein said at least one target gene may be immunoregulatory receptors such as Programmed Death 1 (PD-1, also known as CD279), Cytotoxic T-Lymphocyte Antigen 4 (CTLA-4, also known as CD152), LAG3 (also known as CD223), TIM-3 (also known as HAVCR2), BTLA (also known as CD272), CD160, CD161 (also known as NKR-P1A), T cell immunoreceptor with Ig and ITIM domains (also known as TIGIT), LAIR1 (also known as CD305), Siglec (Sialic acid-binding immunoglobulin-type lectins) such as Siglec3, Siglec7, Siglec9, Siglec10, CD96, KLRG1, ILT2 (also known as LILRB1), CEACAM-1, 2B4, CD100, NTB-A, NKG2A, CD94, Killer Ig-Like Receptors (KIRs), Src-homology 2 domain (SH2)-containing protein tyrosine phosphatases (PTPs) SHP-1 and SHP-2, SH2-containing inositol phosphatase (SHIP), Cytokine-inducible SH2-containing protein (CISH), suppressor of cytokine signaling proteins (SOCS), Ubiquitin thioesterase OTUB1, chemokine receptors such as CCR5, TGF-beta, TGF-beta receptors, ADAM17, or High Affinity Immunoglobulin Epsilon Receptor Subunit Gamma (FcεR1γ), Beta-2 microglobulin (β2M) when said at least one target gene is knocked out.

Said method, wherein said at least one target gene may be (for preventing fratricide) CD38, CLEC12A, CD33, CD123, CD19, CD20, CS1, BCMA, CD56, CD22, CD5, CD7, TIM-3, or LAG-3.

Said method, wherein said at least one target gene may be interleukin-2 (IL-2), IL-2 receptors (IL-2Rs) (alpha, beta and gamma chains), IL-2 with IL-2Rs, IL-2/IL-2R fusion proteins, insulin, IFN-7, IL-4, IL-7, GM-CSF, IL-12, IL-18, IL-18 receptors (IL-18Rs), IL-2/IL-18R fusion proteins, IL-1 family, IL-1 receptors (IL-1Rs), IL-1/IL-1R fusion proteins, IL-15, IL-15 receptors (IL-15Rs), IL-15 with IL-15 receptors (IL-15Rs), IL15/IL-15R fusion proteins, IL-21, IL-21 receptors (IL-21Rs), IL-21 with/IL-21 receptors (IL-21Rs) fusion proteins, IL-33, ST2, IL-33/ST2 fusion protein, CD2, TGFβ, TGFβ receptors and TNF-α or any other additives for the growth of cells, chemokine receptors, chemokines, CD16, NKG2D, NKG2C, NKp44, NKp30, NKp46, when said at least one target one is knocked-in.

Said method, wherein said at least one target gene for the correction of a target gene may be e.g. FCGR3A, GATA2, MCM4, RTEL1, GINS1, and IRF8, IL2RG, ADA.

Said method, wherein said method is performed in a closed system.

Said method, wherein said method is an automated method.

Said method, wherein said method is an automated method in a closed system.

In other aspect the present invention provides a population of genetically engineered NK cells obtained by the method comprising the steps in the following order
a) obtaining a sample comprising NK cells and other cells
b) enrichment of NK cells from said sample
c) introducing a genetic modifier 1 into said NK cells by electroporation
d) introducing a genetic modifier 2 into said NK cells by transduction
e) expanding said genetically modified NK cells.

Said population of genetically engineered NK cells, wherein said population of genetically engineered NK cells expresses a higher level of CD16 compared to a population of genetically engineered NK cells that is obtained by a method that performs first the transduction process of introducing the genetic modifier 2 into the NK cells followed by the electroporation process of introducing the modifier 1 into the NK cells.

Said higher level of expression of CD16 (on the cell surfaces of said genetically engineered NK cells) may be at least 10% higher, 15% higher, 20% higher or 25% higher.

Said population of genetically engineered NK cells, wherein said population of genetically engineered NK cells expresses a higher level of at least one transgene compared to a population of genetically engineered NK cells that is obtained by a method that performs first the transduction process of introducing the genetic modifier 2 into the NK cells followed by the electroporation process of introducing the modifier 1 into the NK cells.

Said higher level of expression of said at least one transgene (on the surface of the genetically engineered NK cells) may be at least 10% higher, 15% higher, or 20% higher or 50% higher and/or said higher level of expression of CD16 (on the surface of the genetically engineered NK cells) may be at least 10% higher, 15% higher or 20% higher, when said population of genetically engineered NK cells expresses at least one transgene as disclosed herein.

Said population of genetically engineered NK cells, wherein said population of genetically engineered NK cells expresses a higher level of CD16 and a higher level of at least one transgene compared to a population of genetically engineered NK cells that is obtained by a method that performs first the transduction process of introducing the genetic modifier 2 into the NK cells followed by the electroporation process of introducing the modifier 1 into the NK cells.

In other aspect the present invention provides a population of genetically engineered NK cells for use in treatment of a disease, wherein said population is obtained by the method comprising the steps in the following order
a) obtaining a sample comprising NK cells and other cells
b) enrichment of NK cells from said sample
c) introducing a genetic modifier 1 into said NK cells by electroporation
d) introducing a genetic modifier 2 into said NK cells by transduction
e) expanding said genetically modified NK cells.

Said disease may be a cancer, an autoimmune disease, an infectious disease or a hereditary disease.

In a further aspect the present invention provides a pharmaceutical composition comprising a population of genetically engineered NK cells obtained by the method comprising the steps in the following order
a) obtaining a sample comprising NK cells and other cells
b) enrichment of NK cells from said sample
c) introducing a genetic modifier 1 into said NK cells by electroporation
d) introducing a genetic modifier 2 into said NK cells by transduction
e) expanding said genetically modified NK cells;
and optionally a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In another aspect the present invention provides a combination of (pharmaceutical) compositions comprising
a) A population of genetically engineered NK cells obtained by the method as disclosed herein, and
b) a therapeutic antibody or fragment thereof comprising a Fc portion or a CD16-engaging domain and optionally
c) a pharmaceutically acceptable carrier.

In another aspect the present invention provides a method for treating a disease in a subject, the method comprising
I) administering to said subject a population of genetically engineered NK cells obtained by the method comprising the steps in the following order
a) obtaining a sample comprising NK cells and other cells
b) enrichment of NK cells from said sample
c) introducing a genetic modifier 1 into said NK cells by electroporation
d) introducing a genetic modifier 2 into said NK cells by transduction
e) expanding said genetically modified NK cells; and
II) administering a therapeutic antibody or fragment thereof comprising a Fc portion or a CD16-engaging domain to said subject.

Said administration of said population of genetically engineered NK cells may be performed before, after or simultaneously to the administration of said therapeutic antibody or fragment thereof comprising a Fc portion or a CD16-engaging domain.

All definitions, characteristics and embodiments defined herein with regard to an aspect of the invention, e.g. the first aspect of the invention, also apply mutatis mutandis in the context of the other aspects of the invention as disclosed herein.

### EMBODIMENTS

In one embodiment of the invention a population of genetically engineered NK cells may be generated, wherein said genetically engineered NK cells express as a transgene a CAR specific for e.g. a tumor associated antigen (TAA) and have been knocked out as target gene an inhibitory receptor such as NKG2A. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with an RNA/Cas9 complex specific for knocking out the NKG2A gene. Subsequently these cells may be transduced with a lentiviral vector comprising a nucleic acid coding for a CAR. The genetically engineered NK cells may be expanded for 14 to 16 days or longer in a cell culture. This may lead to a therapeutically effective population of genetically engineered NK cells.

In one embodiment of the invention a population of genetically engineered NK cells may be generated, wherein said genetically engineered NK cells express as a transgene a CAR specific for e.g. a tumor associated antigen (TAA) and have been knocked out as target gene an inhibitory regulator such as CIS. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with an RNA/Cas9 complex specific for knocking out the CISH gene. Subsequently these cells may be transduced with a lentiviral vector comprising a nucleic acid coding for a CAR. The genetically engineered NK cells may be expanded for 14 to 16 days or longer in a cell culture. This may lead to a therapeutically effective population of genetically engineered NK cells.

In one embodiment of the invention a population of genetically engineered NK cells may be generated, wherein said genetically engineered NK cells express as a transgene a CAR specific for e.g. a tumor associated antigen (TAA) and have been knocked out as target gene a self-antigen such as CLEC12A to avoid NK cell fratricide. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with an RNA/Cas9 complex specific for knocking out the CLEC12A gene. Subsequently these cells may be transduced with a lentiviral vector comprising a nucleic acid coding for a CAR. The genetically engineered NK cells may be expanded for 14 to 16 days or longer in a cell culture. This may lead to a therapeutically effective population of genetically engineered NK cells.

In one embodiment of the invention a population of genetically engineered NK cells may be generated, wherein said genetically engineered NK cells express as a transgene a CAR specific for e.g. a tumor associated antigen (TAA) and have been knocked in as target gene e.g. a cytokine such as IL-15. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with an RNA/Cas9 complex specific for knocking in the TL-15 gene. Subsequently these cells may be transduced with a lentiviral vector comprising a nucleic acid coding for a CAR. The genetically engineered NK cells may be expanded for 14 to 16 days or longer in a cell culture. This may lead to a therapeutically effective population of genetically engineered NK cells.

In one embodiment of the invention a population of genetically engineered NK cells may be generated, wherein said genetically engineered NK cells express as a transgene a high-affinity CD16 (e.g. a CD16 polymorphism) and have been knocked out as target gene an inhibitory receptor such as NKG2A. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with a RNA/Cas9 complex specific for knocking out the NKG2A gene. Subsequently these cells may be transduced with a lentiviral vector comprising a nucleic acid coding for a high-affinity CD16. The genetically engineered NK cells may be expanded for 14 to 16 days or longer in a cell culture. This may lead to a therapeutically effective population of genetically engineered NK cells.

In one embodiment of the invention a population of genetically engineered NK cells may be generated, wherein said genetically engineered NK cells express as transgenes a CAR or TCR specific for e.g. a tumor associated antigen (TAA) together with IL-15 and/or IL-15 receptors, and/or IL-15/IL-15R fusion proteins and have been knocked out as target gene an inhibitory receptor such as NKG2A. This population may be generated by enrichment of NK cells from a blood sample using magnetic cell separation technology such as MACS® using anti-CD56 Microbeads resulting in a sample comprising CD56+ NK cells. These CD56+ NK cells may be electroporated with an RNA/Cas9 complex specific for knocking out the NKG2A gene. Subsequently these cells may be transduced with a lentiviral vector comprising a nucleic acid coding for a CAR. The genetically engineered NK cells may be expanded for 14 to 16 days or longer in a cell culture. This may lead to a therapeutically effective population of genetically engineered NK cells.

In one embodiment of the invention in above-described embodiments the “CAR” may be replaced by a TCR as transgene.

In one embodiment of the invention NK cells are generated that express two or more CARs or a CAR together with a TCR or two or More TCRs.

In one embodiment of the invention NK cells are generated that have been knocked-out for two or more genes and/or knocked-in for two or more genes.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

The term “activation” as used herein refers to inducing physiological changes with a cell that increase target cell function, proliferation and/or differentiation.

The term “transduction” means the transfer of genetic material from a viral agent such as a lentiviral vector particle into a eukaryotic cell such as an NK cell.

The term “electroporation” is a technique in which an electrical field is applied to cells in order to increase the permeability of the cell membrane, allowing chemicals, drugs, protein and/or DNA to be introduced into the cell.

A transgene may be a gene that has been transferred by genetic engineering techniques into a host that normally does not bear this gene. The gene may be a naturally gene that occurs in other cells or may be a recombinant gene. Exemplary transgenes used in the present invention may be the chimeric antigen receptor (CAR) and/or the T cell receptor (TCR).

The T cell receptor (TCR) is a protein complex found on the surface of T cells, or T lymphocytes, that is responsible for recognizing fragments of antigen as peptides bound to major histocompatibility complex (MHC) molecules. However, TCRs can also be non-MHC-restricted, such as most γδ TCRs.

The TCR is composed of two different protein chains (that is, it is a heterodimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes during ontogeny and in diseased states (such as leukemia). Each locus can produce a variety of polypeptides with constant and variable regions.

The term “engineered nuclease” as used herein refers to an endonuclease. The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific nucleic acid sequences, further referred to as “cleavage sites” or “target sequences” or “target sites”.

Engineered nucleases that induces a cleavage at a specific cleavage site of a nucleic acid sequence such as a genome of a cell are well known in the art and e.g. described in WO2018073393 as follows hereunder.

As used herein, the term “TALEN” or “TALE-nucleases” refers to an endonuclease comprising a DNA-binding domain comprising 14-20 or 16-22 TAL domain repeats fused to any portion of the Fok1 nuclease domain.

TALE-nucleases, are fusion protein of a TALE binding domain with a cleavage catalytic domain. These endonucleases have been successfully applied to primary immune cells. Such TALE-nucleases, marketed under the name TALEN, are those currently used to simultaneously inactivate gene sequences in T-cells or other cells originating from donors, in particular to produce allogeneic therapeutic T-cells or other cells such as NK cells in which e.g. the gene encoding TCR (T-cell receptor) is disrupted. TALE-nucleases are very specific reagents because they need to bind DNA by pairs under obligatory heterodimeric form to obtain dimerization of the cleavage domain Fok-1. Left and right heterodimer members each recognizes a different nucleic sequence of about 14 to 20 bp, together spanning target sequences of 30 to 50 bp overall specificity.

Other endonuclease systems derived from homing endonucleases (ex: 1-Onul, or I-Crel), combined or not with TAL-nuclease (ex: MegaTAL) or zing-finger nucleases have also proven specificity, but to a lesser extend so far.

As used herein, the term “meganuclease” refers to an endonuclease that binds double-stranded DNA at a recognition sequence that is greater than 12 base pairs. Preferably, the recognition sequence for a meganuclease is 22 base pairs. A meganuclease can be an endonuclease that is derived from I-Crel, and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art. A meganuclease as used herein binds to double-stranded DNA as a heterodimer or as a “single-chain meganuclease” in which a pair of DNA-binding domains are joined into a single polypeptide using a peptide linker. The term “homing endonuclease” is synonymous with the term “meganuclease.” Meganucleases are substantially non-toxic when expressed in cells, particularly in human T cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be nonidentical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” can refer to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions.

As used herein, the term “CRISPR/Cas” (Clustered Regularly Interspaced Short palindromic Repeats) refers to a caspase-based endonuclease comprising a caspase, such as Cas9, and a guide RNA that directs DNA cleavage of the caspase by hybridizing to a recognition site in the genomic DNA.

Other endonucleases reagents have been developed based on the components of the type II prokaryotic CRISPR (Clustered Regularly Interspaced Short palindromic Repeats) adaptive immune system of the bacteria S. pyogenes. This multi-component system referred to as RNA-guided nuclease system, involves members of Cas9 or Cpf1 endonuclease families coupled with a guide RNA molecules that have the ability to drive said nuclease to some specific genome sequences. Cpf1 is a single RNA-guided endonuclease that provides immunity in bacteria and can be adapted for genome editing in mammalian cells. Such programmable RNA-guided endonucleases are easy to produce because the cleavage specificity is determined by the sequence of the RNA guide, which can be easily designed and cheaply produced. The specificity of CRISPR/Cas9 although stands on shorter sequences than TAL-nucleases of about 10 pb, which must be located near a particular motif (PAM) in the targeted genetic sequence. As used herein, the term “megaTAL” refers to a single-chain nuclease comprising a transcription activator-like effector (TALE) DNA binding domain with an engineered, sequence-specific homing endonuclease.

The term “cleavage” refers to the breakage of the covalent backbone of a nucleic acid sequence (a polynucleotide). As used herein, the cleavage may be initiated or induced by the engineered nuclease as disclosed herein

The term “closed system” as used herein refers to any closed system which reduces the risk of cell culture contamination while performing culturing processes such as the introduction of new material, e.g. by transduction, and performing cell culturing steps such as proliferation, differentiation, activation, separation of cells, and/or electroporation if an in-line electroporation unit is connected. Such a system allows to operate under GMP or GMP-like conditions (“sterile”) resulting in cell compositions which are clinically applicable. Herein exemplarily the CliniMACS Prodigy® (Miltenyi Biotec B. V. & Co. KG, Germany) connected to the CliniMACS® Electroporator (Miltenyi Biotec B. V. & Co. KG, Germany) is used as a closed system. The CliniMACS Prodigy® is disclosed in WO2009/072003. But it is not intended to restrict the use of the method of the present invention to the CliniMACS Prodigy®. The process of the invention may be performed in a closed system, comprising a centrifugation chamber comprising a base plate and cover plate connected by a cylinder, pumps, valves, a magnetic cell separation column and a tubing set. The blood samples or other sources comprising NK cells may be transferred to and from the tubing set by sterile docking or sterile welding. A suitable system is disclosed in WO2009/072003.

The closed system may comprise a plurality of tubing sets (TS) where cells are transferred between TS by sterile docking or sterile welding.

Different modules of the process may be performed in different functionally closed TS with transfer of the product (cells) of one module generated in the one tubing set to another tubing set by sterile means. For example, NK cells can be magnetically enriched in a first tubing set (TS) TS100 by Miltenyi Biotec and the positive fraction containing enriched NK cells is welded off the TS100 and welded onto a second tubing set TS730 by Miltenyi Biotec for further activation, modification, cultivation and washing.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® as disclosed herein.

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising NK cells may be provided in transfer bags or other suited containers which can be connected to the closed system by sterile means.

The term “providing a (cell) sample comprising NK cells” means the provision of a cell sample, preferentially of a human cell sample of hematologic origin. Normally, the cell sample may be composed of hematologic cells from a donor or a patient. Such blood product can be e.g. in the form of whole blood, buffy coat, leukapheresis, PBMCs or any clinical sampling of blood product. It may be from fresh or frozen origin.

The term “washing” means for example the replacement of the medium or buffer in which the cells are kept. The replacement of the supernatant can be in part (example 50% of the medium is removed and 50% fresh medium is added) this often is applied for dilution or feeding purposes, or entirely. Several washing steps may be combined in order to obtain a more profound replacement of the original medium in which the cells are kept. A washing step often may involve pelleting the cells by centrifugation forces and removing the supernatant. In the method of the present invention, cells may be pelleted by rotation of the chamber at e.g. 300×g and the supernatant may be removed during rotation of the chamber. Medium may be added during rotation or at steady state.

Generally, the washing or washing step may be performed once or by a series of media/buffer exchanges (at least twice exchanges, e.g. 2, 3, 4, 5, 6, 7, 8, 9 or 10 exchanges) thereby removing the substances intended to be removed from the immune cells such as NK cells such as human serum and/or its components, the magnetic particles or the residual lentiviral vector particles. The exchanges may be performed by separation of cells and media/buffer by centrifugation, sedimentation, adherence or filtration and subsequent exchange of media/buffer.

For enrichment, isolation or selection in principle any sorting technology can be used. This includes for example affinity chromatography or any other antibody-dependent separation technique known in the art. Any ligand-dependent separation technique known in the art may be used in conjunction with both positive and negative separation techniques that rely on the physical properties of the cells. An especially potent sorting technology is magnetic cell sorting.

Methods to separate cells magnetically are commercially available e.g. from Invitrogen, Stem cell Technologies, in Cellpro, Seattle or Advanced Magnetics, Boston. For example, monoclonal antibodies can be directly coupled to magnetic polystyrene particles like Dynal M 450 or similar magnetic particles and used e.g. for cell separation. The Dynabeads technology is not column based, instead these magnetic beads with attached cells enjoy liquid phase kinetics in a sample tube, and the cells are isolated by placing the tube on a magnetic rack. However, in a preferred embodiment for enriching CD56+ NK cells from a sample comprising NK cells according the present invention monoclonal antibodies or antigen binding fragments thereof are used in conjunction with colloidal superparamagnetic microparticles having an organic coating by e.g. polysaccharides (Magnetic-activated cell sorting (MACS) technology (Miltenyi Biotec B. V. & Co. KG, Germany)). These particles (nanobeads or MicroBeads) can be either directly conjugated to monoclonal antibodies or used in combination with anti-immunoglobulin, avidin or anti-hapten-specific MicroBeads.

The MACS technology allows cells to be separated by incubating them with magnetic nanoparticles coated with antibodies directed against a particular surface antigen. This causes the cells expressing this antigen to attach to the magnetic nanoparticles. Afterwards the cell solution is transferred on a column placed in a strong magnetic field. In this step, the cells attach to the nanoparticles (expressing the antigen) and stay on the column, while other cells (not expressing the antigen) flow through. With this method, the cells can be separated positively or negatively with respect to the particular antigen(s)/marker(s).

In case of a positive selection the cells expressing the antigen(s) of interest, which attached to the magnetic column, are washed out to a separate vessel, after removing the column from the magnetic field.

In case of a negative selection the antibody used is directed against surface antigen(s) which are known to be present on cells that are not of interest. After application of the cells/magnetic nanoparticles solution onto the column the cells expressing these antigens bind to the column and the fraction that goes through is collected, as it contains the cells of interest. As these cells are non-labelled by an antibody coupled to nanoparticles, they are “untouched”.

The procedure can be performed using direct magnetic labelling or indirect magnetic labelling. For direct labelling the specific antibody is directly coupled to the magnetic particle. Indirect labelling is a convenient alternative when direct magnetic labelling is not possible or not desired. A primary antibody, a specific monoclonal or polyclonal antibody, a combination of primary antibodies, directed against any cell surface marker can be used for this labelling strategy. The primary antibody can either be unconjugated, biotinylated, or fluorophore-conjugated. The magnetic labelling is then achieved with anti-immunoglobulin MicroBeads, anti-biotin MicroBeads, or anti-fluorophore MicroBeads.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The tumor associated antigen (TAA) as used herein refers to an antigenic substance produced in tumor cells. Tumor associated antigens are useful tumor or cancer markers in identifying tumor/cancer cells with diagnostic tests and are potential candidates for use in cancer therapy. Preferentially, the TAA may be expressed on the cell surface of the tumor/cancer cell, so that it may be recognized by the antigen binding receptor as disclosed herein.

The term “antigen-binding molecule” as used herein refers to any molecule that binds preferably to or is specific for the desired target molecule of the cell, i.e. the antigen. The term “antigen-binding molecule” comprises e.g. an antibody or antigen binding fragment thereof. The term “antibody” as used herein refers to polyclonal or monoclonal antibodies, which can be generated by methods well known to the person skilled in the art. The antibody may be of any species, e.g. murine, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “antibody” comprises both intact molecules and antigen binding fragments, such as Fab, Fab′, F(ab′)2, Fv, nanobodies and single-chain antibodies. Additionally, the term “antigen-binding fragment” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g. receptor-ligand interaction). The linkage (coupling) between antibody and particle or nanostructure can be covalent or non-covalent. A covalent linkage can be, e.g. the linkage to carboxyl-groups on polystyrene beads, or to NH2 or SH2 groups on modified beads. A non-covalent linkage is e.g. via biotin-avidin or a fluorophore-coupled-particle linked to anti-fluorophore antibody.

The fragment crystallizable region (Fc region) is the tail region of an antibody that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains.

The term “CD16-engaging domains” as used herein may be domains that can specifically recognize and bind to CD16 in order to induce ADCC of NK cells. In addition to a wild-type Fc portion, CD16-engaging domains may be but not restricted to scFvs, Fabs, single variable Fv domains such as VH only, nanobodies, mutated Fc portions with increased or decreased affinity to CD16, and/or Designed Ankyrin Repeat Proteins (DARPins).

The terms “specifically binds to” or “specific for” with respect to an antigen-binding molecule, e.g. an antibody or antigen-binding fragment thereof, refer to an antigen-binding molecule (in case of an antibody or antigen-binding fragment thereof to an antigen-binding domain) which recognizes and binds to a specific antigen in a sample, e.g. CD56, but does not substantially recognize or bind other antigens in said sample. An antigen-binding domain of an antibody or antigen-binding fragment thereof that binds specifically to an antigen from one species may bind also to that antigen from another species. This cross-species reactivity is not contrary to the definition of “specific for” as used herein. An antigen-binding domain of an antibody or antigen-binding fragment thereof that specifically binds to an antigen, e.g. the CD56 antigen, may also bind substantially to different variants of said antigen (allelic variants, splice variants, isoforms etc.). This cross reactivity is not contrary to the definition of that antigen-binding domain as specific for the antigen, e.g. for CD56.

The terms “genetically modified immune cell (NK cell)” or “engineered immune cell (NK cell)” may be used interchangeably and mean containing and/or expressing a foreign gene or nucleic acid sequence which in turn modifies the genotype or phenotype of the cell or its progeny. These terms also may include the gene-knockout, gene knock-in or gene-corrected NK cells generated by endonucleases such as CRISPR/Cas technology are. Especially, the terms refer to the fact that cells can be manipulated by recombinant methods well known in the art to express stably or transiently peptides or proteins, e.g. CARs which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

The term “genetic modifier” as used herein may refer to any material/substance that may be transferable into a eukaryotic cell by methods well-known in the art resulting in a genetic modification of said cell. Preferentially “Genetic modifier 1” may be DNA, RNA, protein, or a combination thereof, more preferentially, the genetic modifier 1 may be DNA, RNA, protein, or a combination thereof able to selectively engineer at least one target gene by DNA cleavage.

The term “genetic modifier 2” may be nucleic acids such as DNA and/or RNA that may be intended to be transduced into a eukaryotic cell. Preferentially, said genetic modifier 2 may be a viral vector. Said viral vector may comprise a transgene. Said viral vector comprising at least one transgene may be a retroviral vector comprising a transgene. Said retroviral vector may be a lentiviral vector.

As used herein “autologous” means that cells, a cell line, or population of cells used for treating subjects are originating from said subject.

As used herein “allogeneic” means that cells or population of cells used for treating subjects are not originating from said subject but from a donor.

The terms “immune cell” or “immune effector cell” refer to a cell that may be part of the immune system and executes a particular effector function such as alpha-beta T cells, NK cells, NKT cells, B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells. Most preferred immune effector cells are T cells and NK cells. “Effector function” means a specialized function of a cell, e.g. in a T cell or NK cell an effector function may be cytolytic activity or helper activity including the secretion of cytokines.

The term “natural killer cells (NK cells)” are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. A subset of human NK cells also express CD8. Continuously growing NK cell lines can be established from cancer patients and common NK cell lines are for instance NK-92, NKL and YTS.

The term “isolated” means altered or removed from the natural state. For example an isolated population of cells means an enrichment of such cells and separation from other cells which are normally associated in their naturally occurring state with said isolated cells. An isolated population of cells means a population of substantially purified cells which are a homogenous population of cells.

As used herein, the term “expansion” or “proliferation” refers to cell growth and multiplication of cell numbers. Expansion or proliferation, as used herein relate to increased numbers of NK cells occurring during the cultivation process.

The terms “culturing process” or “cultivation” as used herein refer to the culturing and expansion of cell such as NK cells. The culturing process may last as long as desired by the operator and can be performed as long as the cell culture medium has conditions which allow the cells to survive and/or grow and/or proliferate.

Herein it is referred to “Elpo-TD method”, if the electroporation step is before the transduction step within the method, and to “TD-Elpo method”, if the transduction step is before the electroporation step within the method. “Elpo method” refers to a method with electroporation only.

Generally, the timeline and timepoints for the Elpo-TD process as disclosed herein may be:
Day0: NK cell enrichment
Day1: Electroporation (e.g. 18 hours after enrichment)
Day2: Transduction (e.g. 48 hours after enrichment)

Further culturing until at least day 15.

Generally, the timeline and timepoints for the TD-Elpo process as disclosed herein may be:
Day0: NK cell enrichment
Day2: Transduction (e.g. 48 hours after enrichment)
Day3: Electroporation (e.g. 72 hours after enrichment)

Further culturing until at least day 15.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells or NK cells that have a natural or genetically engineered reactivity to a patient's cancer are generated in-vitro and then transferred back into the cancer patient. Then the immunotherapy is referred to as “CAR cell immunotherapy” or in case of use of T cells only as “CAR T cell therapy” or “CAR T cell immunotherapy”, when these cells express a CAR.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription and/or translation of a particular nucleotide sequence driven by its promoter in a cell.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

As used herein, the term “expansion” or “proliferation” refers to cell growth and multiplication of cell numbers. Expansion or proliferation, as used herein relate to increased numbers of NK cells occurring during the cultivation process.

In general, a CAR as disclosed herein may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell.

Such a CAR may be also named “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target, i.e. BDCA2. The tag may be e.g. a hapten such as biotin or fluorescein isothiocyanate (FITC) or phycoerythrin (PE), but the tag may also be a peptide sequence e.g. chemically or recombinantly coupled to the polypeptide part of the tagged polypeptide. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA) or the tag of a tagged polypeptide. The CARs of the invention may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, immunoglobulin full length heavy and/or light chains, Fab fragments, single chain Fv (scFv) fragments, nanobodies, single variable fragments such as VH domains, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (or the intracellular signaling domain) of the CAR is responsible for activation of at least one of the normal effector functions of the immune cell in which the CAR is expressed. “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, DAP12, CD5, CD22, CD79a, CD79b, and CD66d. Most prominent is sequence derived from CD3ζ (CD3zeta).

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, 2B4, DAP10.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD).

In some embodiments, the endodomain may contain a primary cytoplasmic signaling domain or a co-stimulatory region, but not both. In these embodiments, an immune effector cell containing the disclosed CAR is only activated if another CAR containing the missing domain also binds its respective antigen.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adaptor molecules could be designed for tumor specificity and would provide options for altering specificity post-adoptive transfer, key for situations of selection pressure and antigen escape.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein.

### EXAMPLES

The following examples are intended for a more detailed explanation of the invention but without restricting the invention to these examples.

### Example 1: CAR Expression and Gene Knockout (KO) Efficiency on CD33-KO CAR-NK Cells Generated by the Electroporation-Transduction (Elpo-TD) Method

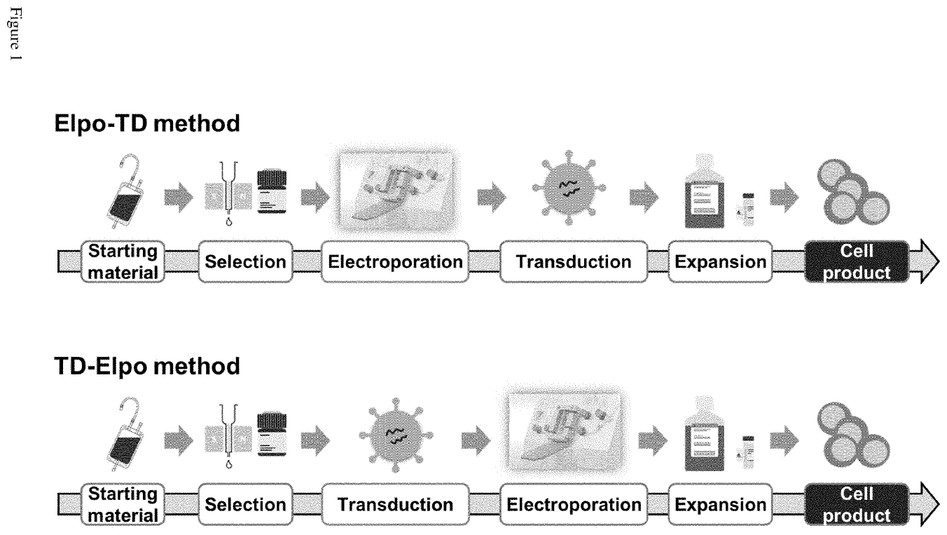

NK cells are enriched from starting materials at day 0 and cultured for 48 hours until viral transduction in both the Electroporation-Transduction (Elpo-TD) and Transduction-Electroporation (TD-Elpo) method. However, NK cells treated with the Elpo-TD method will be electroporated 18 hours after NK cell enrichment but prior to transduction, while NK cells treated with the TD-Elpo method will be electroporated 72 hours after NK cell enrichment but post transduction. Electroporated and transduced NK cells will be further expanded until harvest (FIG. 1).

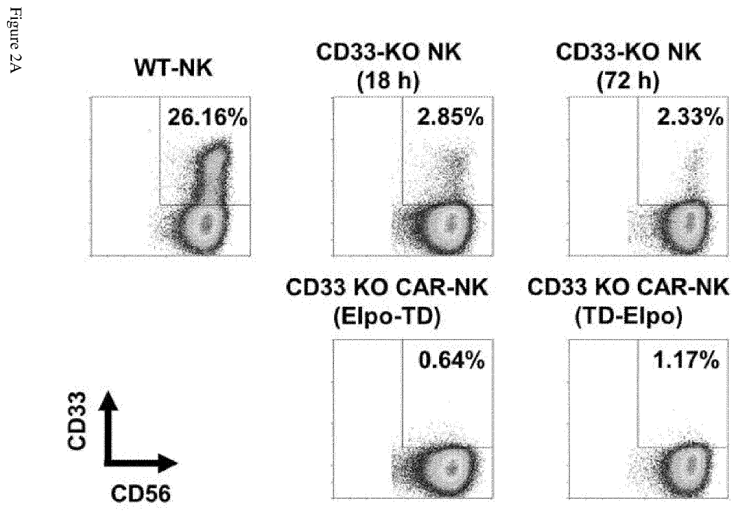

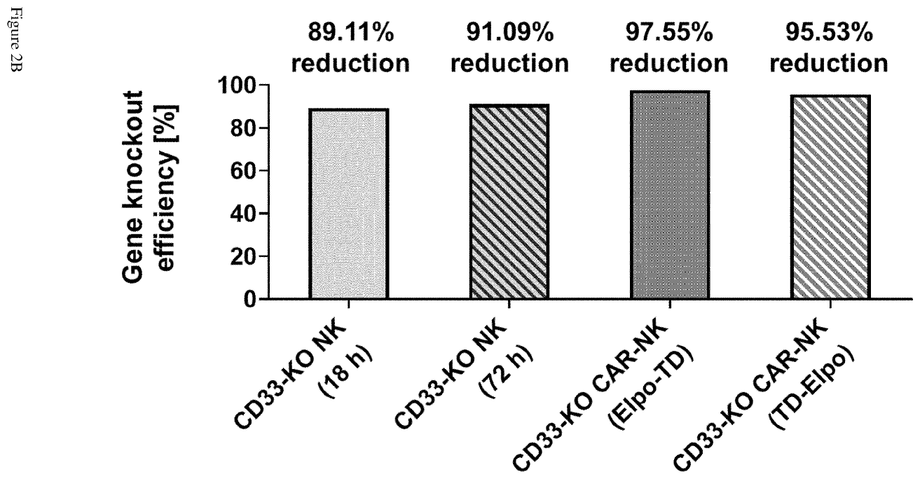

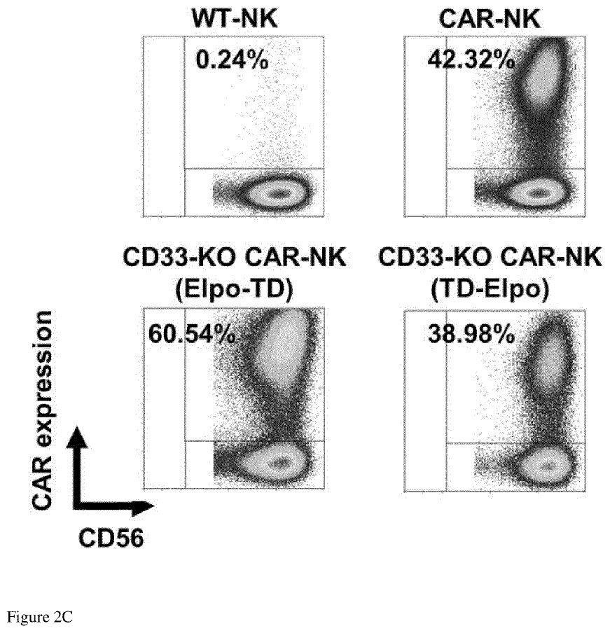

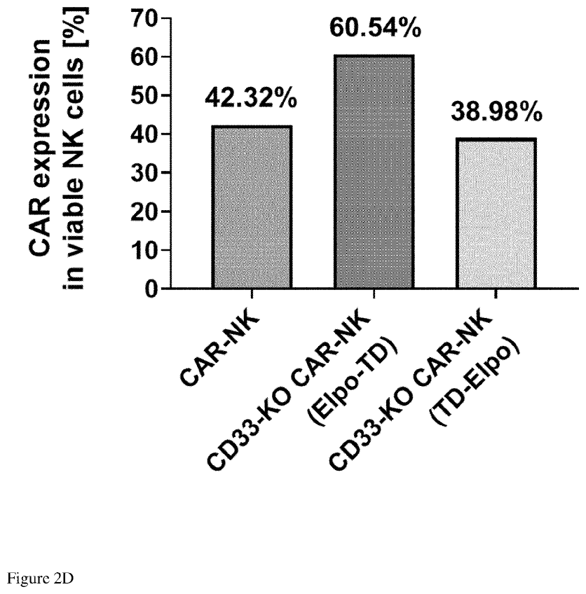

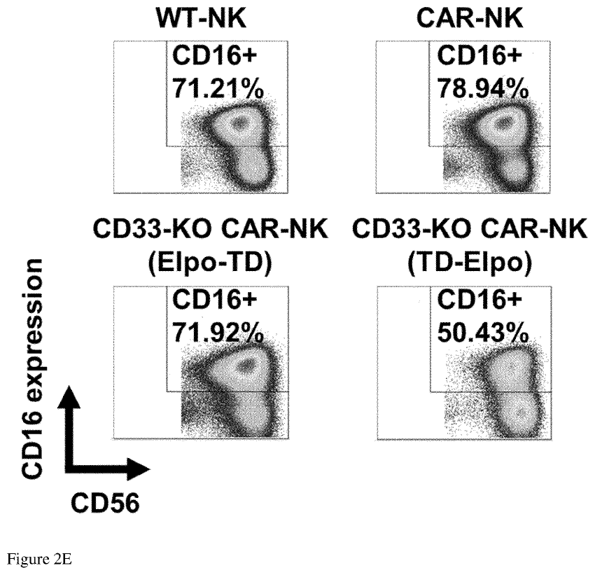

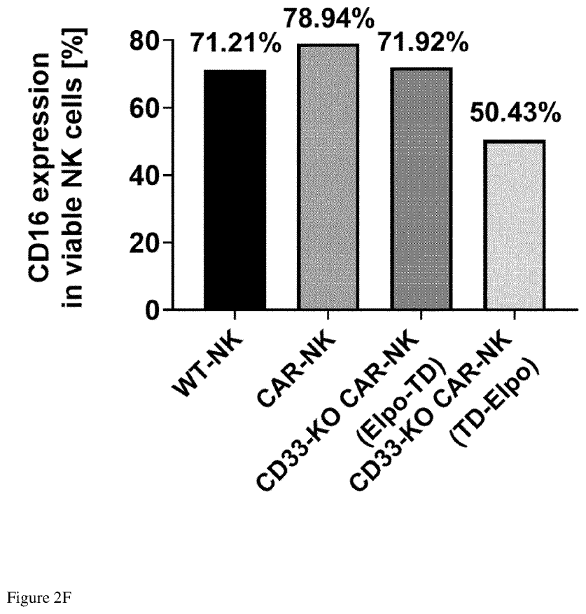

In Example 1, CD33-knockout (KO) CAR-NK cells expressing CD33-specific CARs were generated by either the Elpo-TD method or TD-Elpo method as representative proof-of-concept examples for adoptive therapy. Peripheral blood mononuclear cells (PBMCs) were isolated by Ficoll density gradient centrifugation (Biocoll, Biochrom, Germany). Primary NK cells were enriched from PBMCs at day 0 with CD3-positive cell depletion followed by CD56-positive cell enrichment (positive selection) using antibody coated microbeads (Miltenyi Biotec, Germany), according to the manufacturer's instructions. Isolated NK cells were stimulated and cultured in the complete NK cell culture medium as previously described (Bari et al. (2019). Front. Immunol. 10, 2001). Fourty eight hours after NK cell isolation, NK cells were transduced with Baboon envelope pseudotyped lentiviral vectors (BaEV-LVs) encoding a second generation CD33-specific CAR construct consisting CD33-specific single chain Fv antibody fragment (scFv), human CD8α hinge and transmembrane domain, followed by human CD137 and CD3ζ intracellular domains. The endogenous CD33 expression in NK cells was knocked out with CRISPR (clustered regularly interspaced short palindromic repeats)/Cas (CRISPR-associated) ribonucleoprotein (RNP) complexes (20 μM) of three different guide RNAs (gRNAs) targeting cd33 gene. CD33-specific CRISPR/Cas RNPs were introduced into NK cells either 18 hours (Elpo-TD method) or 72 hours (TD-Elpo method) post-isolation by a CliniMACS Electroporator (Miltenyi Biotec, Germany) with 600 V for 120 s as the first pulse and 200 V for 10 ms as the second pulse. Unmodified wild-type NK (WT-NK) cells, NK cells only receiving electroporation of CRISPR/Cas RNPs targeting cd33 at the corresponding time points (CD33-KO NK) and NK cells only receiving transduction with CD33-specific CARs (CAR-NK) served as controls. The gene-modified NK cells were further expanded in vitro until day 15. CAR expression and gene deletion efficiency in NK cells were determined at day 13. While similarly high CD33 KO efficiency has been achieved by both methods (FIGS. 2A and 2B), higher CAR expression could be detected on the surface of the gene-modified NK cells generated by the Elpo-TD method, compared with those generated by the TD-Elpo method (FIGS. 2C and 2D). Meanwhile, the Elpo-TD method did not impact the surface CD16 expression on gene-modified NK cells, compared with WT-NK cells and CAR-NK cells. However, CD16 expression was markedly reduced on the surface of CAR-NK cells generated the TD-Elpo method (FIGS. 2E and 2F).

CD16, also known as FcγRIIIa, is an activating receptor that binds to Fc portion of antibodies to induce antibody-dependent cell-mediated cytotoxicity (ADCC) of NK cells against tumor cells or viral infected cells. Tumor targeting therapeutic antibodies, such as Rituximab (specific for CD20), Trastuzumab (specific for HER2/ErbB2) or Daratumumab (specific for CD38), have been broadly applied in the clinic with great success. NK cells are critical to clinical responses of therapeutic antibodies by recognizing and eliminating antibody-coated malignant cells through CD16 ligation. It has been demonstrated that increased CD16 expression positively correlated with the degree of ADCC of NK cells and thereby enhances NK cell clinical efficacy (Koehn et al., 2012, Neuroblastoma. Front. Pharmacol. 3; Peruzzi et al., 2013, Human Primary NK Cells. J. Immunol. 191, 1883-1894).

The data of Example 1 demonstrate that the Elpo-TD method combining both electroporation and transduction can be successfully used to generate gene-modified NK cells, resulting in high level of transgene expression and high gene deletion efficiency in NK cells, meanwhile retaining functional NK cell phenotype.

### Example 2: Anti-Tumor Activity of Gene-Modified NK Cells Generated by the Elpo-TD Method

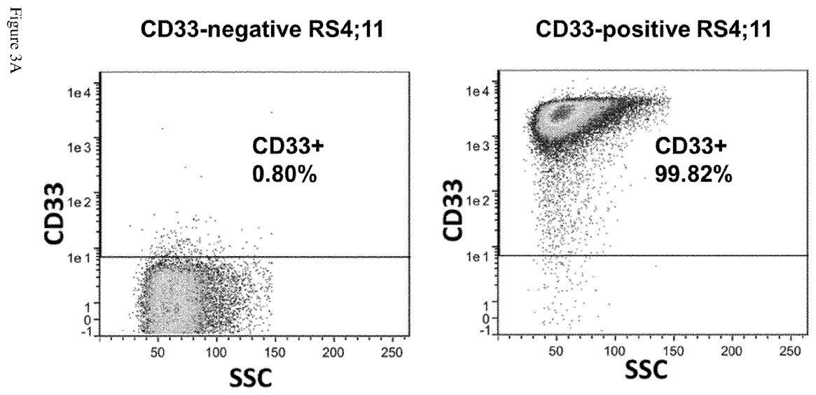

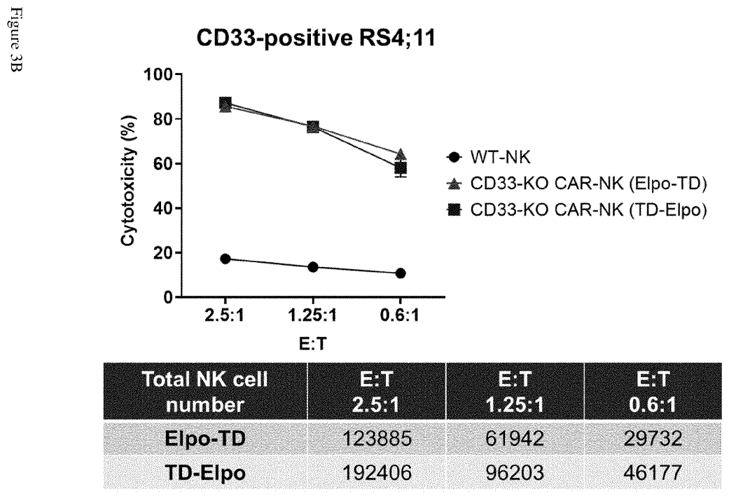

Next, the possible consequences on the anti-tumor activity of gene-KO CAR-NK cells generated by the Elpo-TD method were investigated. Human B cell precursor acute lymphoblastic leukemia (ALL) RS4;11 cells, which are highly resistant towards the natural cytotoxicity of NK cells, were engineered to ectopically express human CD33 on their surface (FIG. 3A). The thereby established CD33-positive RS4;11 cells were used as target cells to determine CAR-mediated NK cell cytotoxicity.

CD33-KO CD33-specific CAR-NK cells generated by either the Elpo-TD or TD-Elpo method were co-cultured with CD33-positive RS4;11 tumor cells for 4 hours at different Effector:Target (E:T) ratios (FIG. 3B). E:T ratios were calculated based on the number of CD33-specific CAR-positive NK cells co-cultured with 30,000 tumor target cells. Unmodified WT-NK cells served as controls. CD33-KO CAR-NK cells generated by both methods exhibited high and specific CAR-mediated cytotoxicity at all E:T ratios examined. However, much less total NK cells derived from the Elpo-TD production method were needed to lyse the same amount of target tumor cells, compared with those derived from the TD-Elpo method (FIG. 3B table). This is benefited from the higher CAR NK cell percentage in the NK cell products generated by the Elpo-TD method, compared with those generated by the TD-Elpo method.

The data summarized in Example 2 demonstrate that the Elpo-TD method can generate gene-modified CAR NK cells bearing high CAR-mediated anti-tumor activity and enables similar tumor cell elimination rate with less total NK cell number from the derived NK cell bulk products.

### Example 3: Proliferation of Gene-Modified NK Cells Generated by the Elpo-TD Method

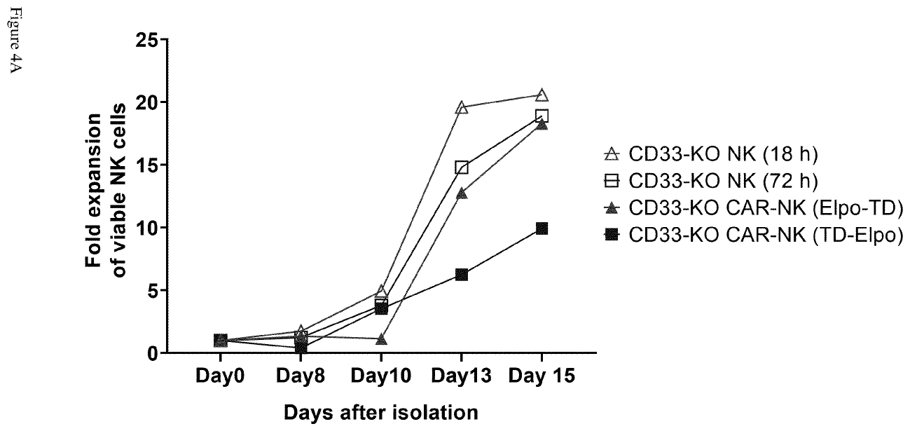

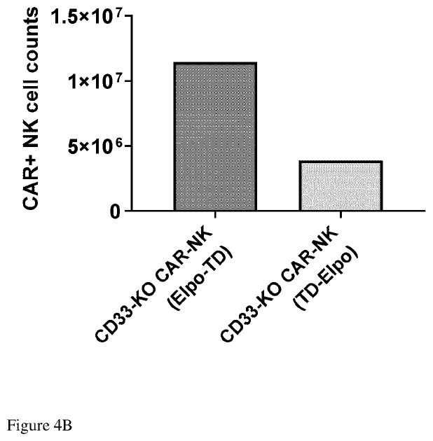

To assess the possible consequence of using the Elpo-TD method to generate gene-modified NK cells on the growth of such cells, cell proliferation was measured over a period of 15 days after NK cell isolation. CD33-KO CAR-NK cells specific for CD33 were cultured in complete NK cell culture medium (NK MACS medium supplemented with 5% human AB serum, 500 IU/mL human IL-2 and 140 IU/mL human IL-15) as previously described (Bari et al. (2019). Front. Immunol. 10, 2001). Half of conditioned culture medium was exchanged in every 2-3 days until the end of the experiment. NK cells only received electroporation of CRISPR/Cas RNPs targeting cd33 gene at the corresponding time points (CD33-KO NK) served as controls. Viable NK cell numbers were counted at the indicated time points. While no marked difference in NK cell expansion could be observed for CD33-KO CAR-NK cells generated by the Elpo-TD method at the end day of the experiment, CD33-KO CAR-NK cells generated by the TD-Elpo method expanded much slower, compared with the control NK cells (FIG. 4A). At the endpoint of the time series (day 15), the Elpo-TD method could generate 2.9 times more CAR-modified NK cells, compared with the TD-Elpo method (FIG. 4B).

The data summarized in Example 3 demonstrate that gene-modified NK cells derived from the Elpo-TD method retain higher proliferation capacity than those derived from the TD-Elpo method.

### Example 4: The Elpo-TD Method is not Restricted to CRISPR RNP Usage

To assess whether the Elpo-TD method is CRISPR RNP-restricted, NK cells were mock electroporated without enzymes or nucleotides, followed by viral transduction with a CD33-specific CAR construct. CD33 CAR NK cells prepared by the TD-Elpo method served as controls. The modified NK cells were expanded in complete NK cell culture medium as described in Example 1.

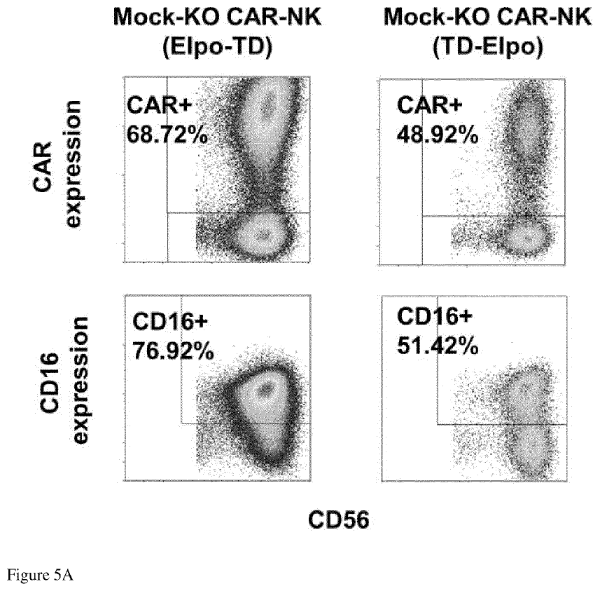

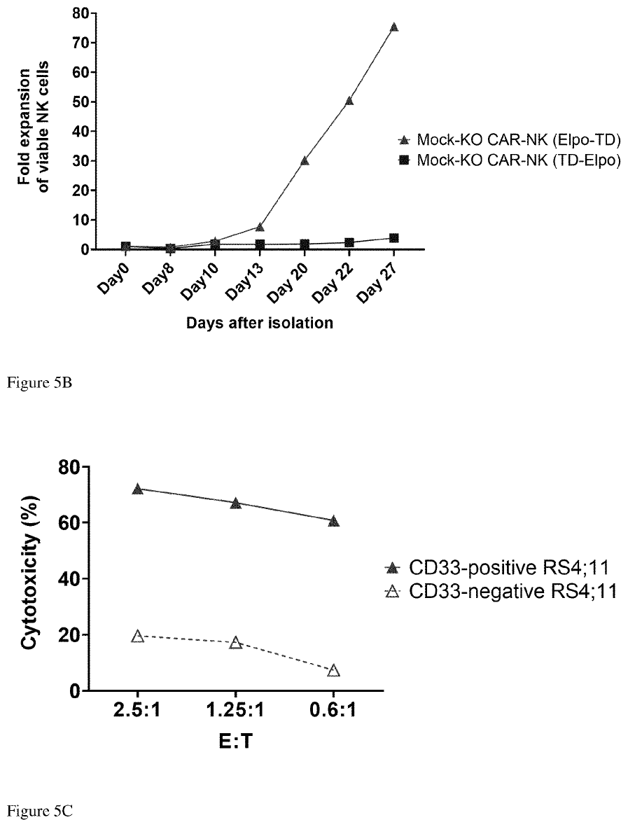

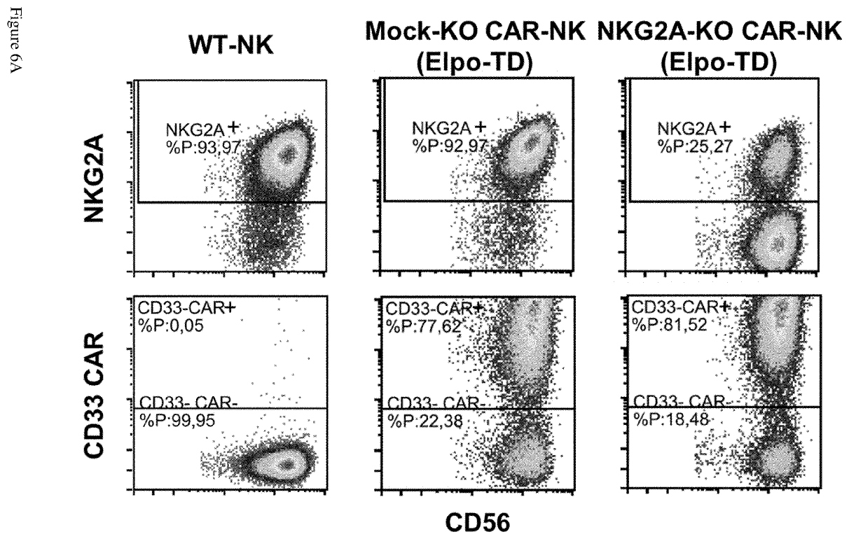

CD33 CAR expression and CD16 expression were determined by flow cytometric analysis at day 13 of in vitro expansion, resulting in higher levels of both CAR and CD16 expression on the surface of CAR-NK cells generated by the Elpo-TD method, compared with those generated by the TD-Elpo method (FIG. 5A). Again, CAR-NK cells generated by Elpo-TD method expanded much faster than those generated by TD-Elpo method, resulting in 75.5-fold and 3.9-fold expansion in the end of the experiment (Day 27), respectively (FIG. 5B). CAR-mediated cytotoxicity of CAR-NK cells generated by the Elpo-TD method was analyzed by co-incubating with CD33-positive or CD33-negative RS4;11 tumor cells for 4 hours at different Effector:Target (E:T) ratios as described in Example 2. E:T ratios were calculated based on the number of CAR-positive NK cells. It showed that CAR-NK cells generated by the Elpo-TD method possess high and specific CAR-mediated cytotoxicity (FIG. 5C).

The results shown in Example 4 are similar to the observation in Example 1, in which CRISPR RNPs targeting CD33 gene were included for electroporation. The data summarized in Example 4 corroborate that using the Elpo-TD method to generate potent gene-modified NK cells for immunotherapy is not restricted to particular proteins or nucleotides, thereby demonstrating the advantages and flexibility of working with the Elpo-TD method to produce potent NK cell therapeutics.

### Example 5: Enhanced Antitumor Activity of NKG2A-KO CAR-NK Cells Generated by the Elpo-TD Method

In another approach NK group 2 member A (NKG2A)-KO CAR-NK specific for CD33 were produced by the Elpo-TD method and characterized in vitro.

NKG2A forms a heterodimeric receptor with CD94 and is an immune checkpoint in NK cells. It can prominently inhibit NK cell activation upon binding to peptide-loaded HLA-E, which is a non-classical class I human leukocyte antigen (HLA) molecule overexpressed in a variety of cancer types (Creelan and Antonia, 2019). Upon cytokine stimulation, NKG2A can be upregulated on NK cells, thereby limiting anti-tumor activity of NK cells in patients (Miller and Lanier, 2019). Therefore, abrogating NKG2A expression in NK cells holds the promise to augment NK cell response in clinical applications.

Primary NK cells were enriched from PBMCs at day 0 and stimulated and cultured in the complete NK cell culture medium as described in Example 1. NKG2A-specific CRISPR RNPs were introduced into NK cells 18 hours post-isolation by a CliniMACS Electroporator (Miletnyi Biotec, Germany) as described in example 1. Fourty eight hours post-isolation, NK cells were transduced with Baboon envelope pseudotyped lentiviral vectors (BaEV-LVs) encoding a second generation CD33-specific CAR construct as described in example 1.

Unmodified wild-type NK (WT-NK) cells and CAR-NK cells electroporated without CRISPR RNPs prior to CAR transduction (Mock-KO CAR-NK) served as controls. The gene-modified NK cells were further expanded in vitro until day 14.

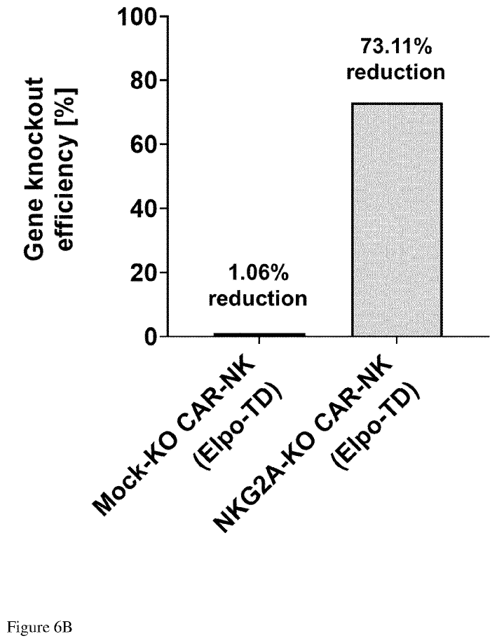

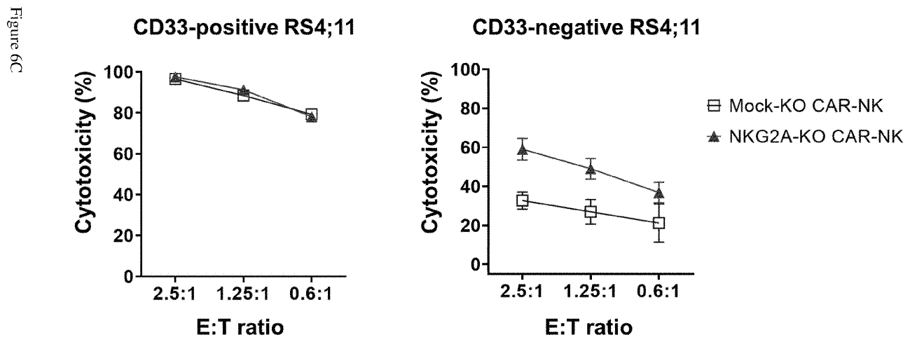

CD33 CAR expression and NKG2A gene deletion efficiency in NK cells were determined at day 13. While similarly high CAR expression has been detected on both Mock-KO and NKG2A-KO CAR-NK cells, NKG2A surface reduction was only obtained for NKG2A-KO CAR-NK cells (FIGS. 6A and 6B). To assess the anti-tumor activity, NKG2A-KO CD33-specific CAR-NK cells generated by the Elpo-TD method were co-cultured with CD33-positive RS4;11 tumor cells for 4 hours at different Effector:Target (E:T) ratios as described in Example 2. Mock-KO CAR-NK cells served as controls. E:T ratios were calculated based on the number of CAR-positive NK cells. While no marked difference in the CD33-specific CAR-mediated cytotoxicity, NKG2A-KO CAR-NK exhibited elevated killing activity against CD33-negative RS4;11 cells, which are otherwise resistant to NK cell natural cytotoxicity partly due to their HLA-E expression (FIG. 6C).

The data summarized in Example 5 demonstrate that the Elpo-TD method can be used to generate CAR NK cells lacking immune checkpoint expression for immunotherapy, thereby promoting their anti-tumor response.

### Example 6: NK Cells Growth within the First Three Days after Isolation

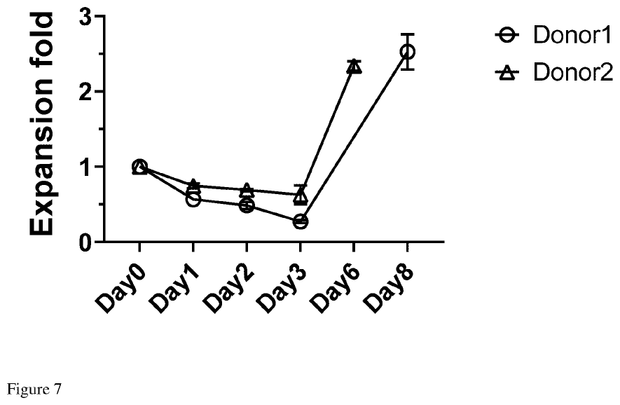

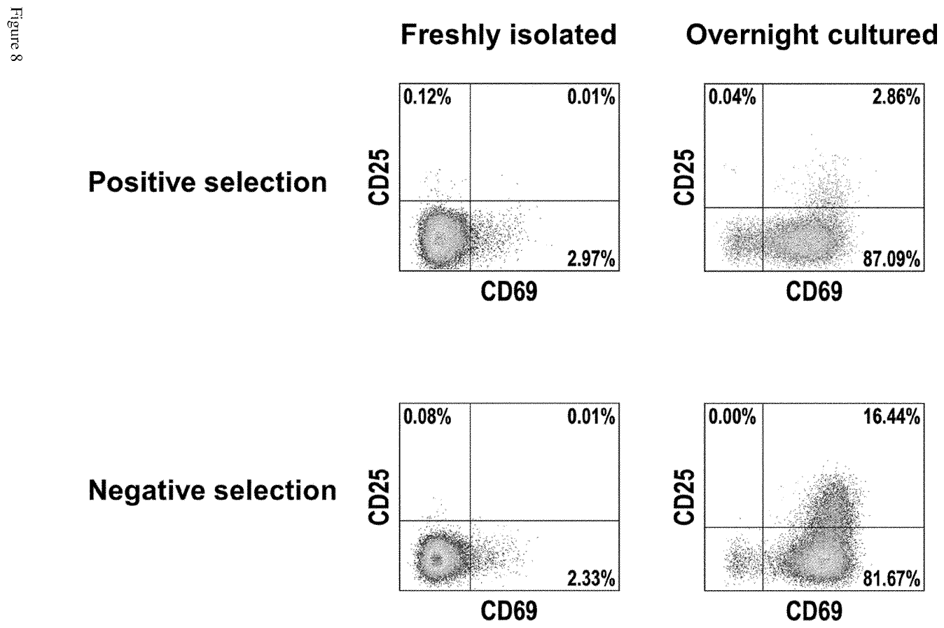

To determine the NK cell growth in culture after isolation, primary NK cells were enriched from PBMCs at day 0 and cultured as described in Example 1. Viable NK cell numbers were counted at the time points indicated in FIG. 7. It showed that the viable NK cell numbers gradually declined in culture from day 0 to day 3, whereas the NK cell numbers increased again in culture at Day 6-8 after isolation (FIG. 7).

The data summarized in Example 6 demonstrate that NK cells do not expand in culture within the first 3 days after isolation.

### Example 7: NK Cells are not Activated During Isolation Upon Positive Selection

To assess whether NK cells will be activated upon CD56 engagement when using CD56 microbeads during cell separation, the activation status of isolated NK cells was determined by flow cytometric analysis. Primary human NK cells were freshly isolated from PBMCs at day 0 with either CD56 microbeads (positive selection) or NK cell isolation kit (negative selection) for human cells (Miltenyi Biotec, Germany). The isolated NK cells were either resuspended in PBS for immediate use or cultured at 37° C. overnight in NK MACS medium supplemented with 5% heat-inactivated human AB serum and 500 IU/mL human IL-2 in an incubator.

The surface expressions of the early activation markers, CD69 and CD25, on the purified NK cells were then determined by flow cytometric analysis. While CD69 and CD25 were upregulated on the NK cells cultured overnight in culture media, no marked expression of these activation markers was detected on the freshly isolated NK cells with either positive or negative selection method (FIG. 8). It showed that both positively and negatively selected NK cells maintain their non-activated (resting) state.

The data summarized in Example 7 demonstrate that NK cells are not activated upon CD56 engagement during the positive selections.

### Example 8: Gene-Modified NK Cells can be Efficiently Generated with the ELPO-TD Method by Performing Electroporation with Freshly Isolated NK Cells

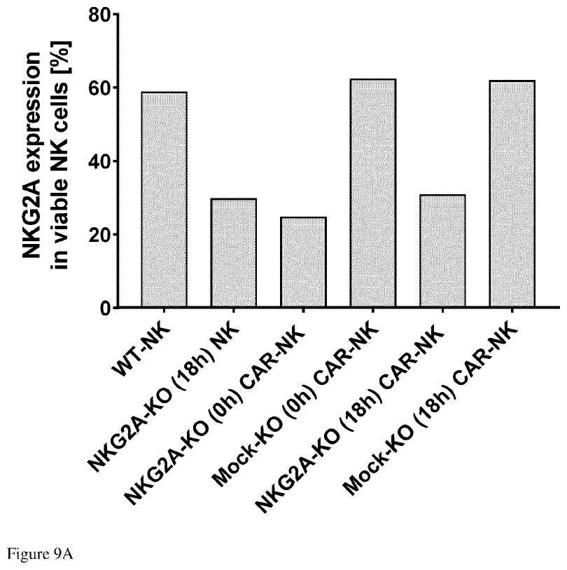

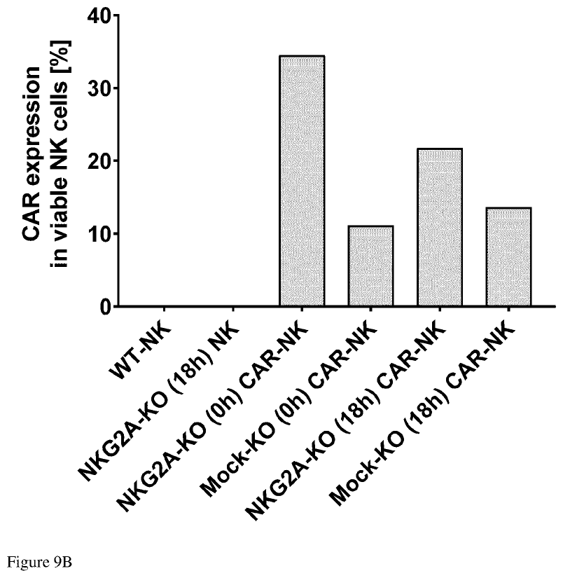

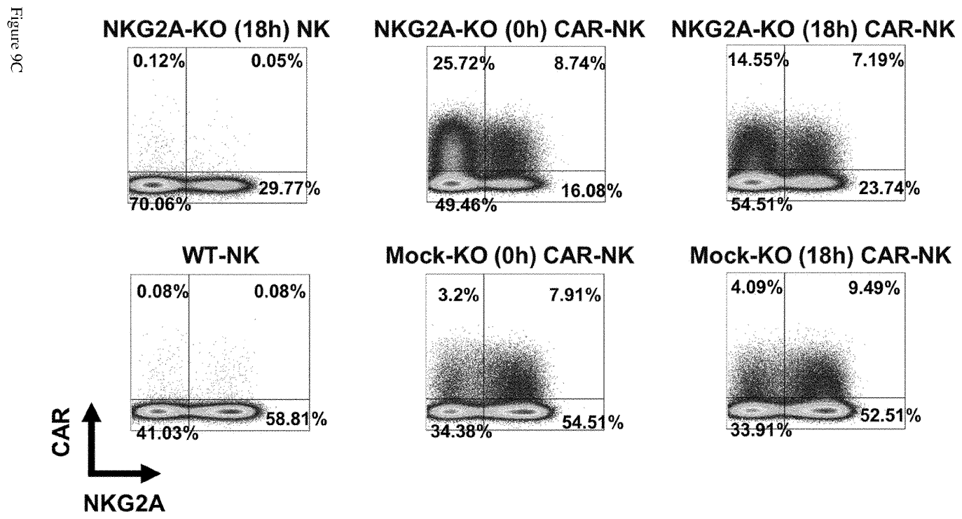

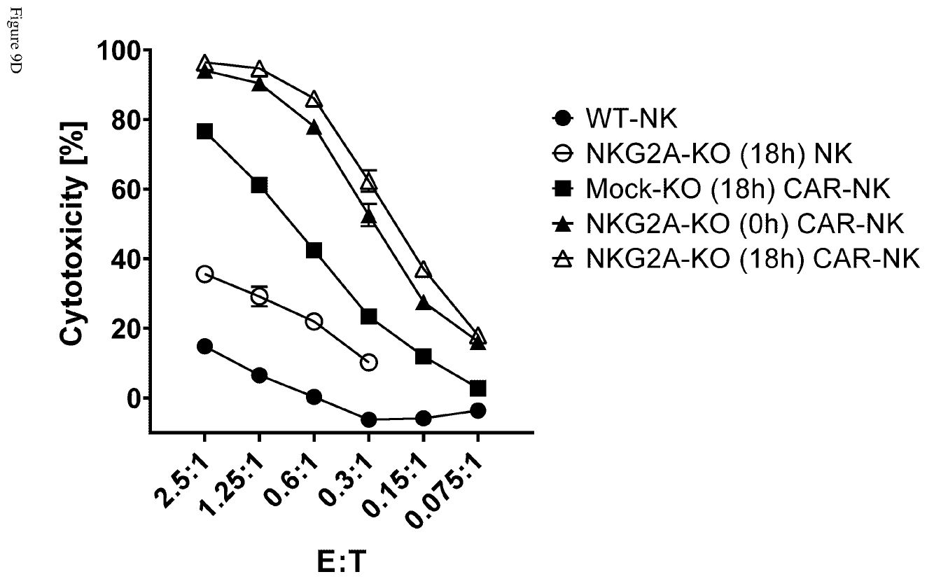

Next, we assessed whether non-activated (resting) NK cells can be efficiently electroporated to generate gene-modified NK cells. Primary NK cells were enriched from PBMCs at day 0 and cultured as described in Example 1. The isolated NK cells were electroporated by a CliniMACS Electroporator (Miltenyi Biotec, Germany) with NKG2A-specific CRISPR RNPs (NKG2A-KO) or without any RNPs (Mock-KO) as described in Example 1. The electroporation was performed with either freshly isolated NK cells 0 hour (0 h) after preparation of the sample and direct subsequent NK cell enrichment or overnight-cultured NK cells 18 hours (18 h) after preparation of the sample and direct subsequent NK cell enrichment. Fourty eight hours after preparation of the sample and direct subsequent NK cell enrichment, the electroporated NK cells were transduced with Baboon envelope pseudotyped lentiviral vectors (BaEV-LVs) encoding a second generation blood dendritic cell antigen 2 (BDCA2)-specific CAR construct consisting of BDCA2-specific scFv, human CD8α hinge and transmembrane domain, followed by human CD137 and CD3ζ intracellular domains. The generated gene-modified NK cells were indicated as NKG2A-KO CAR-NK or Mock-KO CAR-NK in FIG. 9. Unmodified wild-type NK (WT-NK) cells and overnight-cultured NK cells only electroporated with NKG2A-specific CRISPR RNPs 18 hours after preparation of the sample and direct subsequent NK cell enrichment (NKG2A-KO (18 h) NK) served as controls.

The gene-modified NK cells were further cultured in vitro until day 15. CAR expression and NKG2A gene deletion efficiency in NK cells were determined at day 13. A similarly high NKG2A knockout efficiency was achieved for the NK cells electroporated 0 hour after preparation of the sample and direct subsequent NK cell enrichment followed by CAR transduction (NKG2A-KO (0 h) CAR-NK), compared with those electroporated 18 hours after preparation of the sample and direct subsequent NK cell enrichment followed by CAR transduction (NKG2A-KO (18 h) CAR-NK) or those only electroporated with NKG2A-specific CRISPR RNPs 18 hours after preparation of the sample and direct subsequent NK cell enrichment (NKG2A-KO (18 h) NK) (FIG. 9A). No NKG2A reduction was observed for Mock-KO CAR-NK cells, compared with WT-NK cells (FIG. 9A). The BDCA2 CARs could only be detected on the surface of the NK cells transduced with the CAR construct (FIG. 9B). The dot plots of the NKG2A and CAR expression for all the samples are shown in FIG. 9C. The anti-tumor activity of the gene-modified NK cells was further investigated. RS4;11 tumor cells, which are highly resistant towards the natural cytotoxicity of NK cells, were engineered to ectopically express human BDCA2 on their surface, resulting in RS4;11/BDCA2 tumor cells to serve as target cells. The gene-modified NK cells generated in Example 8 were co-cultured with RS4;11/BDCA2 target cells for 16 hours at different Effector:Target (E:T) ratios. E:T ratios were calculated based on the number of CAR-positive NK cells. Unmodified WT-NK cells served as controls. While NKG2A-KO (18 h) NK cells and Mock-KO (18 h) CAR-NK cells showed higher anti-tumor activity compared with WT-NK cells, the anti-tumor activity was further enhanced for both NKG2A-KO (0 h) CAR-NK cells and NKG2A-KO (18 h) CAR-NK cells, which exhibited similarly high cytotoxicity against the target tumor cells (FIG. 9D). It shows that the downregulation of NKG2A expression can further unleash the anti-tumor potential of CAR NK cells.

The data summarized in Example 8 demonstrate that gene-modified NK cells can be efficiently generated with the ELPO-TD method by performing electroporation with freshly isolated NK cells. Such gene-modified NK cells possess high therapeutic potentials.

### Example 9: Determination of Time Frames to Perform Electroporation to Generate Gene-Modified NK Cells by Using the ELPO-TD Method

Next, we determined suitable time frames to perform electroporation to generate gene-modified NK cells by using the ELPO-TD method.

Primary NK cells were enriched from PBMCs at day 0 and cultured as described in Example 1. The isolated NK cells were electroporated by a CliniMACS Electroporator (Miltenyi Biotec, Germany) with NKG2A-specific CRISPR RNPs (NKG2A-KO) as described in Example 1. The NK cell electroporation was performed immediately (0 h), 18 hours (18 h), 48 hours (48 h) or 144 hours (144 h) after preparation of the sample and direct subsequent NK cell enrichment. Fourty eight hours after preparation of the sample and direct subsequent NK cell enrichment, the NK cells electroporated 0 h, 18 h and 48 h after preparation of the sample and direct subsequent NK cell enrichment were then transduced with BaEV-LVs encoding the second generation CD33-specific CAR as described in Example 1, resulting in NKG2A-KO (0 h) CAR-NK cells, NKG2A-KO (18 h) CAR-NK cells and NKG2A-KO (48 h) CAR-NK cells, respectively. For the NK cells electroporated 144 h after preparation of the sample and direct subsequent NK cell enrichment, the transduction with the CD33-specific CAR was performed 24 hours after electroporation, resulting in NKG2A-KO (144 h) CAR-NK cells. Unmodified wild-type NK (WT-NK) cells and NK cells only transduced with the CD33-specific CAR construct 48 hours after preparation of the sample and direct subsequent NK cell enrichment (CAR-NK) served as controls.

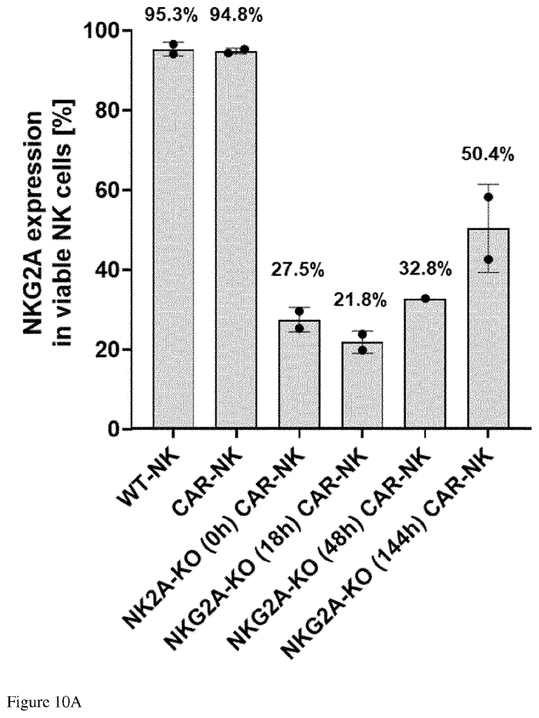

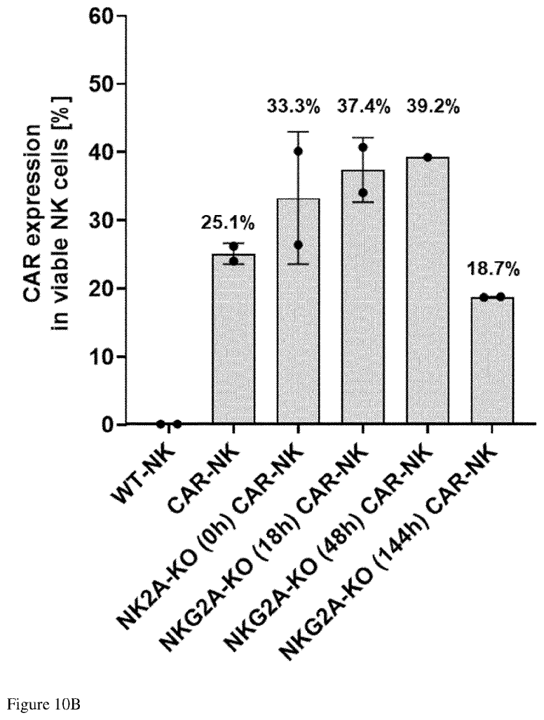

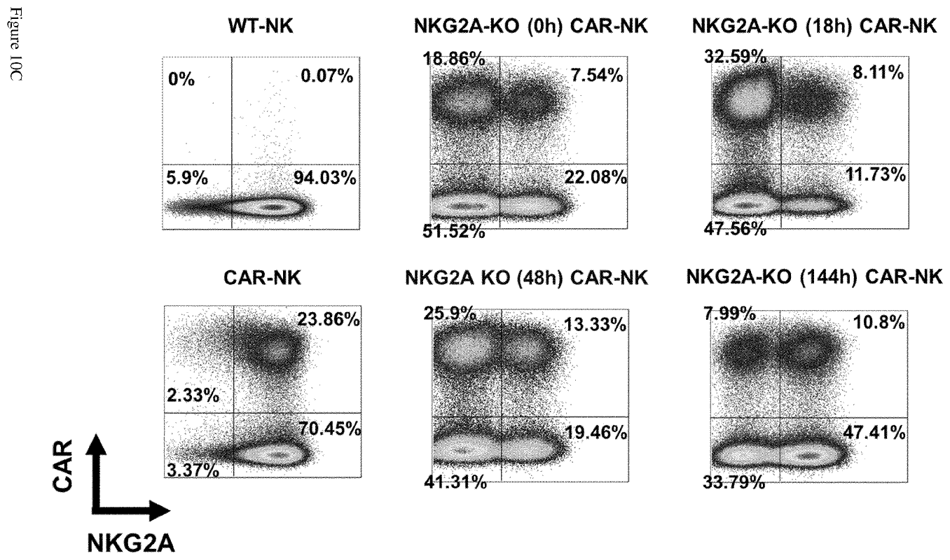

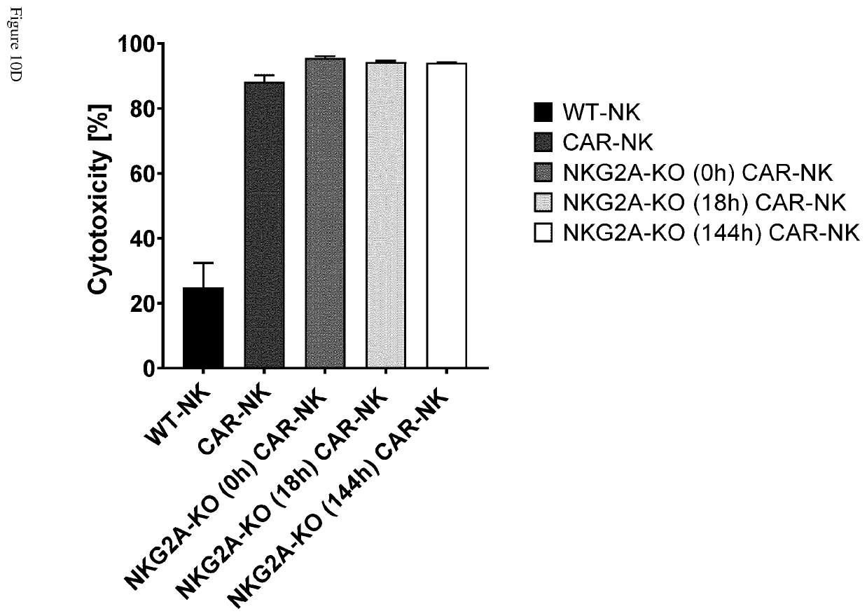

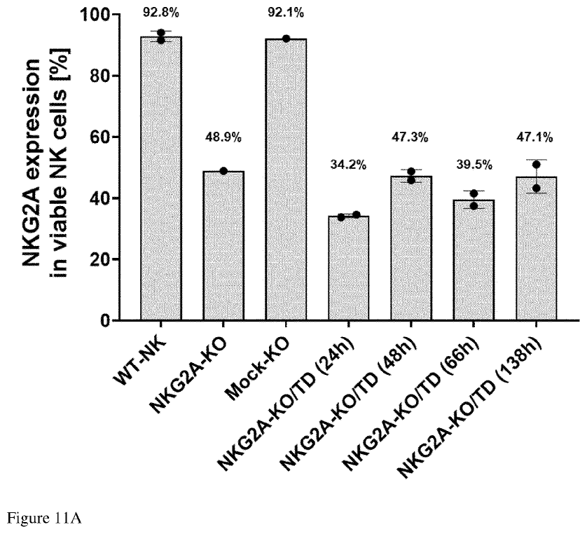

The gene-modified NK cells were further cultured in vitro until day 15. CAR expression and NKG2A gene deletion efficiency in NK cells were determined at day 13. Although the reduction of the NKG2A expression has been detected for all the samples engineered with the NKG2A-specific CRISPR RNPs, the NKG2A gene deletion efficiency was higher for the CAR NK cells electroporated between 0 h and 48 h after preparation of the sample and direct subsequent NK cell enrichment, compared with the CAR NK cells electroporated 144 h after preparation of the sample and direct subsequent NK cell enrichment (FIG. 10A). Moreover, NKG2A-KO (0 h) CAR-NK cells, NKG2A-KO (18 h) CAR-NK cells and NKG2A-KO (48 h) CAR-NK cells showed higher CAR expression, compared with the CAR NK cells only transduced with the CAR construct but without any electroporation (CAR-NK, FIG. 10B). This observation is similar to the results shown in the previous examples (FIG. 2D). However, the enhanced CAR expression was not shown for NKG2A-KO (144 h) CAR-NK cells, compared with CAR-NK cells (FIG. 10B). The dot plots of the NKG2A and CAR expression for the samples from one representative donor are shown in FIG. 10C.

The anti-tumor activity of the gene-modified NK cells was further investigated. CD33-positive OCI-AML2 acute myeloid leukemia (AML) cells served as target cells. The gene-modified NK cells were co-cultured with OCI-AML2 target cells for 18 hours at an Effector:Target (E:T) ratio of 1:1. The E:T ratio was calculated based on the number of CAR-positive NK cells. Unmodified WT-NK cells served as controls. All the NK cells transduced with the CAR construct efficiently lysed the target tumor cells, compared with WT-NK cells (FIG. 10D). The data summarized in Example 9 demonstrate that gene-modified NK cells can be more efficiently generated using the ELPO-TD method when electroporation is performed at the earlier time points rather than at the later time points after preparation of the sample and direct subsequent enrichment of the NK cells.

### Example 10: Determination of Time Intervals Between Electroporation and Transduction to Generate Gene-Modified NK Cells by Using the ELPO-TD Method

We further determined suitable time intervals between electroporation and transduction to generate gene-modified NK cells by using the ELPO-TD method.

Primary NK cells were enriched from PBMCs at day 0 and cultured as described in Example 1. The isolated NK cells were electroporated 18 hours (18 h) after preparation of the sample and direct subsequent NK cell enrichment by a CliniMACS Electroporator (Miltenyi Biotec, Germany) with NKG2A-specific CRISPR RNPs as described in Example 1. The electroporated NK cells were then transduced 6 hours, 30 hours, 48 hours or 120 hours after electroporation with BaEV-LVs encoding the second generation CD33-specific CAR as described in Example 1, resulting in NKG2A-KO/TD (24 h), NKG2A-KO/TD (48 h), NKG2A-KO/TD (66 h), and NKG2A-KO/TD (138 h) CAR-NK cells, respectively. Unmodified wild-type NK (WT-NK) cells and NK cells only electroporated 18 hours (18 h) after preparation of the sample and direct subsequent NK cell enrichment with NKG2A-specific CRISPR RNPs (NKG2A-KO) or without any RNPs (Mock-KO) served as controls.

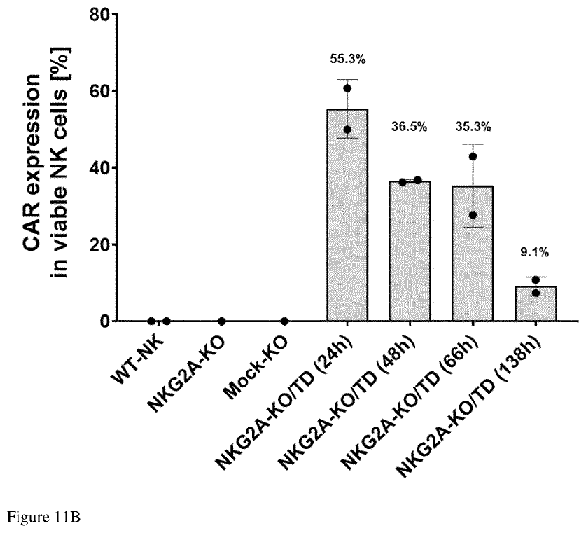

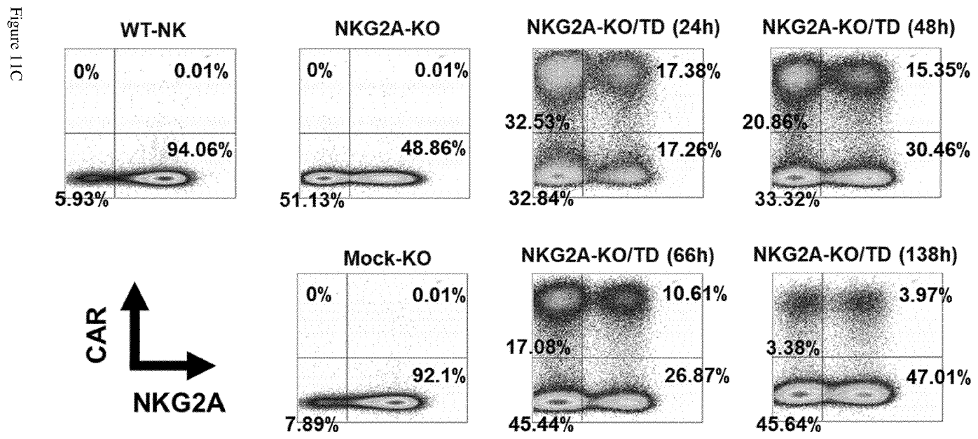

The gene-modified NK cells were further cultured in vitro until day 15. CAR expression and NKG2A gene deletion efficiency in NK cells were determined at day 13. Similarly, high NKG2A knockout efficiencies were achieved for the NK cells electroporated with NKG2A-specific CRISPR RNPs followed by transduction with the CD33-specific CAR construct, compared with NKG2A-KO NK cells (FIG. 11A). No NKG2A reduction was observed for Mock-KO NK cells, compared with WT-NK cells (FIG. 11A). The CAR expression can only be detected on the surface of the NK cells transduction with the CAR construct, although higher CAR expression was shown for the NK cells transduced between 6 h and 48 h after electroporation (FIG. 11B). The dot plots of the NKG2A and CAR expression for the samples from one representative donor are shown in FIG. 11C.

The data summarized in Example 10 demonstrate that gene-modified NK cells can be generated by using the ELPO-TD method with different time intervals between electroporation and transduction.

1-12. (canceled)

13. An in vitro method for the generation of a population of genetically modified natural killer (NK) cells, comprising the steps in the following order:
a) obtaining a sample comprising NK cells and other cells,
b) enriching NK cells from said sample,
c) introducing a genetic modifier 1 into said NK cells by electroporation,
d) introducing a genetic modifier 2 into said NK cells by transduction, and
e) expanding said genetically modified NK cells,
whereby said genetic modifier 1 and said genetic modifier 2 together introduce at least one transgene into the genome of said NK cells, thereby generating a population of genetically modified NK cells.

14. The method according to claim 13, wherein said method comprises before said enrichment of NK cells a step of preparation of said sample,
wherein said electroporation is performed between 0 hours and 48 hours after said preparation of the sample, and
wherein said transduction is performed between 6 hour and 48 hours after said electroporation.

15. The method according to claim 13, wherein said genetic modifier 2 is a viral vector comprising at least one transgene, and wherein said genetic modifier 1 comprises or encodes an enzyme that cleaves genomic DNA in the NK cells, and thereby catalyzes introduction of the transgene(s) in said viral vector into the genome of the NK cells.

16. The method according to claim 15, wherein said genetic modifier 1 is an engineered nuclease selected from the group consisting of a meganuclease, a zinc-finger nuclease (ZFN), a transcription activator-like effector nuclease (TALE-Nuclease), a CRISPR/Cas nuclease, MAD nuclease, CRISPR/Cpf1, Cas12-type-derived nucleases, and a megaTAL nuclease.

17. The method according to claim 15, wherein said genetic modifier 1 is a CRISPR ribonuclear protein (RNP).

18. The method according to claim 15, wherein said genetic modifier 2 is a pseudotyped lentiviral vector.

19. The method according to claim 13, wherein the method generates at day 15 after steps (a) to (d) at least two times more genetically modified NK cells in said population compared with a method that comprises introducing the genetic modifier 2 into the NK cells before introducing the modifier 1 into the NK cells, wherein in both methods the starting amount of NK cells in said sample is the same.

20. The method according to claim 15, wherein said at least one transgene is a chimeric antigen receptor (CAR) or a T-cell receptor (TCR).

21. The method of claim 20, wherein said CAR or TCR is specific for an antigen normally expressed by NK cells, and wherein the transgene is introduced into the genome of the NK cell at a locus that before being genetically modified encodes said antigen, thereby limiting fratricide of the genetically modified NK cells.

22. The method of claim 21, wherein said antigen that is normally expressed by NK cells is a cluster of differentiation (CD) antigen selected from CD38, CLEC12A, CD33, CD123, CD19, CD20, CS1, BCMA, CD56, CD22, CD5, CD7, TIM-3, and LAG-3

23. The method of claim 21, wherein said antigen that is normally expressed by NK cells is CD33.

24. The method according to claim 13, wherein said method is performed in a closed system.

25. The method according to claim 24, wherein said method is an automated method.

26. A population of genetically engineered NK cells obtained according to the method of claim 13.

27. The population of genetically engineered NK cells according to claim 26, which express at least 10% more CD16, compared with a reference population of genetically engineered NK cells obtained by introducing the genetic modifier 2 into the NK cells before introducing the genetic modifier 1 into the NK cells.
